(19) United States
(12) Patent Application Publication
(10) Pub. No.: US 2009/0227503 A1
(43) Pub. Date: Sep. 10, 2009

(54) PARATHYROID HORMONE TREATMENT FOR ENHANCED ALLOGRAFT AND TISSUE-ENGINEERED RECONSTRUCTION OF BONE DEFECTS

(75) Inventors: Hani A. Awad, Rochester, NY (US); Edward M. Schwarz, Rochester, NY (US); Susan Bukata, Pittsford, NY (US); J. Edward Puzas, Pittsford, NY (US); Regis J. O'Keefe, Rochester, NY (US); Randy Rosier, Rochester, NY (US)

Correspondence Address: MCDERMOTT WILL & EMERY LLP, 18191 VON KARMAN AVE., SUITE 500, IRVINE, CA, 92612-7108, US

(73) Assignee: UNIVERSITY OF ROCHESTER, Rochester, NY (US)

(21) Appl. No.: 12/368,251

(22) Filed: Feb. 9, 2009

Related U.S. Application Data
(60) Provisional application No. 61/027,006, filed on Feb. 7, 2008.

(51) Int. Cl. A61K 38/29; A61P 19/00

(52) U.S. Cl. 514/12

(57) ABSTRACT

Methods of improving an outcome of a bone allograft procedure in a subject suffering from a massive bone defect are described, including providing a bone allograft to the subject and intermittently providing said subject with parathyroid hormone (PTH); where the PTH is provided in an amount effective to enhance in or adjacent to a bone allograft, relative to a patient not provided the PTH, at least one of callus bone volume, callus mineral content, callus bridging, graft stiffness, graft incorporation, and graft resistance to an applied torque.

### RELATED APPLICATIONS

This application claims priority to U.S. Provisional Patent Application No. 61/027,006, filed on Feb. 7, 2008, the contents of which are hereby incorporated by reference in their entirety.

### GOVERNMENT RIGHTS

This invention was made with government support under grant numbers NIH R01AR51469 and NIH P50 AR054041, awarded by the National Institutes of Health, and grants from the Musculoskeletal Transplant Foundation, the Wallace H. Coulter Foundation. The U.S. government has certain rights in this invention.

### FIELD OF THE INVENTION

Some embodiments of the invention relate to methods of tissue-engineered reconstruction of bone defects; more specifically, some embodiments of the invention relate to methods of parathyroid hormone (PTH)—or related naturally-occurring peptides or recombinant forms thereof—treatment for enhanced allograft incorporation and tissue-engineered reconstruction of massive bone defects.

### BACKGROUND OF THE INVENTION

Structural bone allografts are widely used in orthopaedics to fill critically sized defects. However, their impaired incorporation, remodeling and high failure rates limit allografts' survival half-life. Adjuvant therapies such as coengraftment with mesenchymal stem cells, local gene therapy, and various chemical and mechanical treatments have been applied for initializing the revitalization of bone allografts with varying success. In addition numerous studies have investigated the use of synthetic biomaterial scaffolds in lieu of bone allografts for tissue-engineered reconstruction of massive long bone defects but have to report clinically relevant success.

### SUMMARY OF THE INVENTION

Thus, there exists a need for methods of enhancing graft-host incorporation, and improving biomechanical strength of both allograft-reconstructed and tissue-engineered reconstruction of massive structural bone defects. In some embodiments, an allograft for a massive bone defect may comprise an axial length of at least 3 cm, at least 4 cm, at least 5 cm, at least 6 cm, at least 7 cm, at least 8 cm, at least 9 cm, and at least 10 cm. Some embodiments of the invention satisfy this need and provide related advantages as well.

Some embodiments of the invention provide a method of improving an outcome of a bone allograft procedure in a subject suffering from a massive bone defect, including providing a bone allograft to the subject and intermittently providing said subject with parathyroid hormone (PTH); where the PTH is provided in an amount effective to enhance in or adjacent to a bone allograft, relative to a patient not provided the PTH, at least one of callus bone volume, callus mineral content, callus bridging, graft stiffness, graft incorporation, and graft resistance to an applied torque.

Certain embodiments provide method, of improving an outcome of a bone graft procedure in a patient, comprising: providing a bone graft to a patient; and intermittently providing a parathyroid hormone (PTH) to the patient in an amount effective to enhance in or adjacent to the bone graft, relative to a patient not provided the PTH, at least one of callus bone volume, callus mineral content, callus bridging, graft stiffness, graft incorporation, and graft resistance to an applied torque.

In certain embodiments, an effective amount is provided by daily injection of the PTH in an amount of at least about 0.1 mg/kg body weight/day. In certain embodiments, an effective amount is provided by daily injection of the PTH in an amount of at least about 0.4 mg/kg body weight/day.

In certain embodiments, the PTH is provided for a period of at least 4 weeks.

In certain embodiments, the PTH comprises native PTH. In certain embodiments, the PTH comprises PTH (1-34).

In certain embodiments, callus bone volume is increased by at least about 75%. In certain embodiments, bone mineral content is increased by at least about 50%. In certain embodiments, ultimate torque is increased by at least about 60%.

In certain embodiments, the bone graft provided comprises an autograft. In certain embodiments, the bone graft provided comprises an allograft.

In certain embodiments, the PTH is effective to increase bone stiffness. In certain embodiments, the PTH is effective to increase bone brittleness. In certain embodiments, the PTH is effective to result in at least one of a reduced risk of pre-union and early union failure of a graft. In certain embodiments, the PTH is provided in an amount effective to enhance callus bone volume in or adjacent to the bone allograft, relative to a patient not provided the PTH. In certain embodiments, the PTH is provided in an amount effective to enhance callus mineral content in or adjacent to the bone allograft, relative to a patient not provided the PTH. In certain embodiments, the PTH is provided in an amount effective to enhance callus bridging in or adjacent to the bone allograft, relative to a patient not provided the PTH. In certain embodiments, the PTH is provided in an amount effective to enhance graft stiffness, relative to a patient not provided the PTH. In certain embodiments, the PTH is provided in an amount effective to enhance incorporation of the bone allograft, relative to a patient not provided the PTH. In certain embodiments, the PTH is provided in an amount effective to enhance graft resistance to an applied torque, relative to a patient not provided the PTH.

### BRIEF DESCRIPTION OF THE DRAWINGS

FIG. 1 shows a representative volume-rendered micro CT images of structural cortical bone grafts 6 weeks after implantation into animals that received saline or PTH systemically as compared to the control animals receiving saline.

FIG. 2 shows a tissue engineered scaffold for structural bone reconstruction. Panel (A) is a low-power SEM of the cross-section of a PLA/βTCP (PLA) scaffold (Scale bar 100:0 represents 1 mm). Panels B and C show high power SEM images of the PLA and 85:15 PLA/βTCP scaffolds respectively. Titanium pins were passed through the lumen of the scaffolds (panel D) to be used for fixation of the scaffolds when implanted as standalone femoral graft substitutes in critical 4 mm femoral defects in a mouse model (panel E).

FIG. 3 shows Micro-CT rendering of the effects of scaffold type and PTH treatment on bone regeneration. Representative micro-CT segmentation of the mineralized callus in femurs reconstructed with PLA scaffolds (A & C) or PLA/βTCP scaffolds (B & D) at 9 weeks post-reconstruction are shown in this figure.

FIG. 4 shows the effects of PTH treatment on the volume of the mineralized callus in two scaffold types. Quantitative micro-CT segmentation of the mineralized callus volume at 6 weeks (A) and at 9 weeks (B) are shown. Panel (C) shows the callus volume of specimens that developed a bridging union compared to non-union control and PTH-treated specimens. Data are presented as mean+SEM. Asterisk indicates significant differences from control (p<0.05).

FIG. 5 shows the prototypical bone torsion behavior of bridged or non-bridged grafts in animals treated or not with PTH.

FIG. 6 shows the effects of PTH treatment on biomechanical properties of the scaffold-grafted femurs especially in femurs with bridging unions. The scaffold-grafted femurs were tested in torsion to determine their biomechanical properties, including maximum torque (A&B), torsional rigidity (C&D), and ultimate normalized rotation or twist (E&F).

FIG. 7 illustrates an exemplary application of an embodiment of a graft-to-host Union Ratio algorithm.

FIG. 8 illustrates an algorithm validation using a digital model.

FIG. 9 illustrates representative micro-CT sagittal sections of 6 and 9 week allografts and autografts (9A) with the corresponding union area maps and Union Ratio numerical values (9B).

FIG. 10 illustrates an application of a multivariable linear regression analysis of geometric micro-CT-based parameters including bone volume, polar moment of inertia (PMI), and Union Ratio.

FIG. 11 illustrates an application of a multivariable linear regression analysis of geometric micro-CT-based parameters including bone volume, polar moment of inertia (PMI), and Union Ratio for allografts only.

FIG. 12 illustrates an estimation of a Union Area from clinical CT data of human patients.

FIG. 13 illustrates the experimental design of an embodiment of a bone allograft and hrPTH treatment.

FIG. 14 illustrates graphs of linear regressions between mechanical properties and Union Ratio of bone grafts, according to certain embodiments of the invention.

FIG. 15 illustrates graphs of multivariable linear regression of certain bone graph and PTH treatment embodiments.

### DETAILED DESCRIPTION OF THE INVENTION

The bone-anabolic effect of systemically administered PTH has been shown to enhance bone mineral density for the treatment of osteoporosis clinically (Talalaj, 2006; Whitfield, 2006; Cosman, 2006), and has recently been shown to enhance fracture repair in animal studies and human patientsm (Chalidis, 2007; Manabe, 2007; unpublished anecdotal observations from the co-inventor Dr. Bukata) but has never been examined in the context augmenting structural allograft healing or in the context of tissue engineering bone biomaterial substitutes for massive structural bone defects.

Prior to the present invention, massive allografts used to repair critically-sized bone defects, from, e.g., tumors or trauma, commonly experience complications due to incomplete graft-host osseointegration which leads to persistent non-union. Furthermore, fatigue fractures due to accumulation and propagation of microdamage within the graft tissue might lead to catastrophic failure. As a result, up to half of large structural cortical allografts in children receiving allografts after bone tumor resection fail in the first 5 years of the life of the graft. Therefore, the development of adjuvant therapies to improve the longevity of the allografts, as provided by certain embodiments of the present invention, will have a tremendous impact on the patients' quality of life and the economic burden of this problem. Prior to the present invention, however, there did not exist accepted quantifiable and non-invasive outcome measures of improved biomechanical strength for allograft healing in patients that could allow for the reliable evaluation of the functional efficacy of these approaches in reasonably-sized clinical trials. In pre-clinical animal models, the standard assay for functional outcome measures is the destructive evaluation of the biomechanical properties. But such evaluation is not possible in a clinical setting.

Before such outcome measures can be used in clinical applications, they would first have to be developed and validated in pre-clinical animal models. To that end, we utilize a mouse model of femoral reconstruction to investigate the differences in the biomechanics of live autograft and devitalized allografts. Using micro computed tomography (micro-CT), we observed that devitalized allograft remodeling and incorporation into the host remained severely impaired compared to live autografts mainly due to the extent of callus formation around the graft and the rate and extent of the graft resorption.

Accordingly, some embodiments of the invention provide a micro-CT based algorithm to compute a 3D measure of union between host (bone and callus) and graft (autograft or allograft) based on the surface area of the graft onto which bone forms to connect the graft to the host. The ratio of connected graft area to total graft surface area can be computed for each graft end and the lesser value for each graft is termed the Union Ratio. This technique can be useful for investigating variation in the osseointegration of femoral bone grafts to determine whether the Union Ratio significantly correlates with the torsional strength and rigidity of bone allografts.

Accordingly, some embodiments of the invention relate to a method for increasing bone formation and graft incorporation by administering a parathyroid hormone (PTH) and/or PTH related peptides or derivatives to a subject with massive bone defects. The method can be employed to improve union between the host and graft/scaffold and to increase bone stiffness and/or toughness or brittleness at the reconstructed site of a massive bone trauma.

Massive bone trauma or defects generally include surgical and mechanical trauma to bone, massive long bone defect, bone defects or gaps larger than 2 cm in length, iatrogenic resection of large segments of bone, osteosarcoma or the like. Increasing bone toughness and/or stiffness generally includes increasing mineral density of cortical bone, increasing callus bone volume, increasing callus mineral content, improving callus bridging, increasing strength of bone, increasing resistance to loading, increasing resistance to rotational force, increasing graft stiffness, improving graft incorporation, increasing graft resistance to an applied torque and the like.

As used herein, the term “parathyroid hormone (PTH)” refers to PTH, teriperitide, PTH related peptides (PTHrP), or PTH derivatives. PTH or PTH related peptides or derivatives are active ingredients in a PTH composition or solution used in the methods of some embodiments of the invention. PTH can be the full length, 84 amino acid form of parathyroid hormone, particularly the human form, hPTH (1-84), obtained either recombinantly, by peptide synthesis or by extraction from human fluid. See, for example, U.S. Pat. No. 5,208,041, incorporated herein by reference. The amino acid sequence for hPTH (1-84) is reported by Kimura et al. in Biochem. Biophys. Res. Comm., 114(2):493.

The PTH can further include ingredient fragments or variants of fragments of human PTH or of rat, porcine or bovine PTH that have human PTH activity. The parathyroid hormone fragments desirably incorporate at least the first 28 N-terminal residues, such as PTH(1-28), PTH(1-31), PTH(1-34), PTH(1-37), PTH(1-38) and PTH(1-41). Alternatives in the form of PTH variants incorporate from 1 to 5 amino acid substitutions that improve PTH stability and half-life, such as the replacement of methionine residues at positions 8 and/or 18 with leucine or other hydrophobic amino acid that improves PTH stability against oxidation and the replacement of amino acids in the 25-27 region with trypsin-insensitive amino acids such as histidine or other amino acid that improves PTH stability against protease. Other suitable forms of PTH used in the methods of some embodiments of the invention include PTHrP, PTHrP(1-34), PTHrP(1-36) and analogs of PTH or PTHrP that activate the PTH1 receptor. These forms of PTH are embraced by the term “parathyroid hormone” as used generically herein. The hormones may be obtained by known recombinant or synthetic methods, such as described in U.S. Pat. Nos. 4,086,196 and 5,556,940, incorporated herein by reference.

The preferred hormone is human PTH(1-34), also known as teriparatide. Stabilized solutions of human PTH(1-34), such as recombinant human PTH(1-34) (rhPTH(1-34), that can be employed in the present method are described in U.S. patent application Ser. Nos. 09/555,476; 10/055,509; 10/427,259; 11/541,862; and 11/541,863; incorporated herein by reference. Crystalline forms of human PTH(1-34) that can be employed in the present method can also be obtained (as Forteo) from Eli Lilly and Company®, Indianapolis, Ind.

A parathyroid hormone-related peptide (PTHrP) is a protein which is known to exist in at least three isoforms of 139, 141 and 173 amino acids. Karaplis et al., Genes & Developme, 8:277-289 (1994). PTHrP is highly homologous to the N-terminal fragment of parathyroid hormone (PTH), and binds the same receptor as PTH. PTHrP appears to play a substantial role in calcium metabolism by an autocrine/paracrine mechanism, and also appears to regulate embryonal development, vascular tone and nutrition. Tsukazaki et al., Calcif Tissue Int 57:196-200 (1995).

The nucleotide and amino acid sequences of the PTHrP gene from rat, mouse and human are known and may be used to produce PTHrP-like polypeptides useful in some embodiments of the invention. See Karaplis et al., Mol. Endocrin. 4:441-446 (1990) [rat]; Mangin et al., PNAS 85:597-601 (1988) [human] and Mangin et al., Gene 95:195-202 (1990) [mousel; and Martinet al., Crit. Rev Biochem Mal Biol 26:377-395 (1991).] In some embodiments of the invention, a variant of PTHrP is used in which one or more amino acids from the carboxy terminus have been deleted. For example, PTHrPI-34, which comprises the first 34 amino acids of PTHrP, is used in some embodiments of the of the invention. Also useful are PTH-like polypeptides which are equivalent to PTHrPI-34 in their ability to enhance survival of chondrocytes. Such PTH-like polypeptides may include, for example, PTH, whether of human, porcine, bovine or other mammalian origin; variants of PTH, such as those described in Wingender et al., U.S. Pat. No. 5,455,329; Wingender et al., U.S. Pat. No. 5,457,047; and Schluter et al., U.S. Pat. No. 5,457,092, and the references cited therein; as well as variants of the above in which one or more amino acids of PTH has been deleted from the carboxy and/or amino terminal portions of the molecule. The disclosures of the above publications are hereby incorporated by reference. PTH, PTHrP and the above variants may be produced via recombinant DNA engineering using the known sequences of the PTH and PTHrP proteins, or may be isolated by purification.

As used herein, the “stiffness” refers to the slope of the linear portion of a load-deformation curve. Stiffness can be measured and calculated by methods standard in the bone study art. These parameters are structural properties that depend on intrinsic material properties and geometry, and can be determined as described in Turner C H, Burr D B., “Basic biomechanical measurements of bone: a tutorial.” Bone 14:595-608 (1993), which is incorporated herein by reference. Ultimate force, stiffness, and work to failure can be normalized to obtain intrinsic material properties such as ultimate stress, elastic modulus, and toughness or brittleness which are independent of size and shape. The ultimate stress refers to maximum stress that a specimen can sustain; elastic modulus refers to material intrinsic stiffness; and as used herein, the terms “toughness” and “brittleness” refer to resistance to fracture per unit volume and the post-yield deformation, respectively. Each of these can be determined by methods known in the art. Id. The strength of a bridge in a femoral graft, for example, can be measured at the reconstructed site typically using three-point or four-point bending at the site or torsion testing.

Accordingly, some embodiments of the invention provide a method for reconstructing bone defects in a subject by using processed allografts and/or synthetic biomaterial tissue engineering scaffolds fixed by standard surgical hardware (plates, nails, rods, external fixators, etc) and procedures and administration of an effective amount of PTH, teriperitide; a PTH derivative, PTH related peptides (PTHrP), and/or other drugs and growth factors.

In one aspect, some embodiments of the invention provide a method for improving the outcome of a bone allograft procedure in a subject treated with a systemic, intermittent administration of an effective amount of PTH, PTH related peptides or derivatives relative to a subject not receiving such a treatment. In one embodiment, an effective amount of PTH, teriperitide; a PTH derivative, PTH related peptides (PTHrP), and/or other drugs and growth factors can be administered intermittently (e.g., irregularly during a day or week), regularly (e.g., once or more each day or week), or cyclically (e.g., regularly for a period of days or weeks followed by a period without administration). In another embodiment, an effective amount of PTH, teriperitide; a PTH derivative, PTH related peptides (PTHrP), and/or other drugs and growth factors can be administered before or after the surgical reconstructive procedure and up to several weeks following the procedure.

Some embodiments are directed to a method of reconstructing massive bone defects in a subject using tissue engineering biomatrials/scaffolds, processed allografts, autografts, or demineralized bone matrix and administering to said patient an intermittent systemic dosage of parathyroid hormone (PTH), teriperitide; a PTH derivative, a PTH related peptides (PTHrP), and/or other drugs and growth factors.

In another aspect, some embodiments of the invention are directed to a method of reducing bone graft rejection in a subject or incidence of fracture at the site of bone and graft/scaffold union by treating the subject with an intermittent systemic dosage of parathyroid hormone (PTH), teriperitide; a PTH derivative, a PTH related peptides (PTHrP), and/or other drugs and growth factors (e.g. VEGF, BMPs) compared to an untreated control population. Some embodiments are directed to a method of reducing the risk of pre-union and/or early union failure of a graft by treating the subject with an intermittent systemic dosage of parathyroid hormone (PTH), teriperitide; a PTH derivative, a PTH related peptides (PTHrP), and/or other drugs and growth factors.

Bone graft materials of some embodiments of the invention include autograft, allograft, demineralized bone matrix (DBM) and tissue engineering biomaterials/scaffolds. A bone graft as an implant allows excellent postoperative imaging because it does not cause scattering like metallic implants on CT or MRI imaging. Autografts can be the ideal form of bone grafts but are available in only limited quantities, since they must be surgically recovered from another location in the subject. Many synthetic bone grafts include materials that closely mimic mammalian bone, such as compositions containing calcium phosphates. Exemplary calcium phosphate compositions contain type-B carbonated hydroxyapatite [Ca5(PO—) (CCb)x(OH)], which is the principal mineral phase found in the mammalian body. The ultimate composition, crystal size, morphology, and structure of the body portions formed from the hydroxyapatite are determined by variations in the protein and organic content. Calcium phosphate ceramics have been fabricated and implanted in mammals in various forms including, but not limited to, shaped bodies and cements. Different stoichiometric compositions such as hydroxyapatite (HAp), tricalcium phosphate (TCP), tetracalcium phosphate (TTCP), and other calcium phosphate salts and minerals, have all been employed to match the adaptability, biocompatibility, structure, and strength of natural bone. The role of pore size and porosity in promoting revascularization, healing, and remodeling of bone has been recognized as a critical property for bone grafting materials. The preparation of exemplary porous calcium phosphate materials that closely resemble bone have been disclosed, for instance, in U.S. Pat. Nos. 6,383,519 and 6,521,246, incorporated herein by reference in their entireties.

In order to possess bulk properties suitable for the graft material to take on load, the compositions used to fabricate the bone grafts preferably include a continuous, biodegradable polymer phase, with the biodegradable wax component initially being dispersed substantially homogenously there through and the biodegradable inorganic filler. The individual components may be blended together such that the wax is homogeneously dispersed through the polymer phase. Such blends then may be further processed by standard methods of compounding, for example extrusion or batch compounding, followed by chopping of the compounded material to form pellets and the like of the homogenous blend. The pellets then may be used to prepare medical devices according to some embodiments of the invention, for example, by extrusion or compression molding, where the fabrication of the medical device from the compounded compositions either includes or is followed by a heat treatment step.

Alternately, the individual components may be added directly to a compounding and molding apparatus, for example an extruder having the proper mixing screw configuration so as to homogenously blend the components in the extrusion barrel, with the extruder being fitted with the appropriate die and heating elements to form bone grafts used in the methods of some embodiments of the invention. A person skilled in the art is able to select the proper parameters and specific apparatus required for the particular blend of components and medical device being fabricated.

The continuous polymer phase comprises a high molecular weight, biocompatible, biodegradable polymer. High molecular weight polymers include polymers with an inherent viscosity (IV) of greater than about 2.0 dl/g when measured in chloroform at 25° C. A biodegradable polymer used in the fabrication of a bone graft of some embodiments of the invention can be degraded or otherwise broken down in the body such that the components of the degraded polymer may be absorbed by or otherwise passed from the body.

Examples of suitable biocompatible, biodegradable polymers that could be used according to some embodiments of the invention include, without limitation, polymers selected from the group consisting of aliphatic polyesters, poly(amino acids), copoly(ether-esters), polyalkylenes oxalates, polyamides, poly(ethylene glycol), poly(iminocarbonates), polyorthoesters, polyoxaesters, polyamidoesters, polyoxaesters containing amine groups, poly(anhydrides), polyphosphazenes, biopolymers, and copolymers and blends thereof.

Aliphatic polyesters include, but are not limited to, homopolymers and copolymers of lactide (which includes lactic acid, D-, L- and meso lactide), glycolide (including glycolic acid), epsilon-caprolactone, para-dioxanone (1,4-dioxan-2-one), trimethylene carbonate (1,3-dioxan-2-one), alkyl derivatives of trimethylene carbonate, monoglyceride polyesters, and polymer blends thereof.

Preferred polymers utilized in some embodiments of the invention comprise homopolymers of lactide (PLA) and homopolymers of glycolide (PGA). More preferred are copolymers of PLA and PGA (PLGA), such copolymers comprising from about 80 to about 99 mole percent PLA.

The wax component of some embodiments of the invention is a low molecular weight biocompatible, biodegradable polymer with a low coefficient of friction. Low molecular weight polymers as defined herein comprise polymers with an Inherent Viscosity (IV) of less than about 0.7 dl/g when measured in chloroform at 25° C. Preferably, the IV is between about 0.3 and 0.5 dl/g when measured in chloroform at 25° C.

Examples of suitable biocompatible, biodegradable waxes that could be used include, without limitation low molecular weight polymers selected from the group consisting of aliphatic polyesters, poly(amino acids), copoly(ether-esters), polyalkylenes oxalates, polyamides, poly(ethylene glycol), poly(iminocarbonates), polyorthoesters, polyoxaesters, polyamidoesters, polyoxaesters containing amine groups, poly(anhydrides), polyphosphazenes, biopolymers, and copolymers and blends thereof.

Aliphatic polyesters which can be made into a wax include, but are not limited to, homopolymers and copolymers of lactide (including lactic acid, D-, L- and meso lactide), glycolide (including glycolic acid), ε-caprolactone, para-dioxanone (1,4-dioxan-2-one), trimethylene carbonate (1,3-dioxan-2-one), alkyl derivatives of trimethylene carbonate, monoglyceride polyesters, and blends thereof.

For example, monoglyceride polyester (MGPE) materials suitable for some embodiments of the invention include biocompatible, biodegradable aliphatic polyester waxes made by the polycondensation of monoalkanoyl glycerides and common dicarboxylic acids These MGPEs have an aliphatic polyester backbone with pendant fatty acid ester groups and exhibit relatively low melting points, e.g. less than about 100° C. Preferred waxes preferably have a melting point of below about 80° C., more preferably from about 45° C. to about 60° C.

Among the preferred wax materials are copolymers of lactide (PLA) and glycolide (PGA) (PLGA); epsilon-caprolactone (PCL) and lactide (PCLA); and epsilon-caprolactone and para-dioxanone (PDO) (PCDO). A preferred wax material is a copolymer of 95 mole percent PCL and about 5 mole percent PDO (95/5 PCDO).

The most preferred wax material comprises a copolymer of epsilon-caprolactone and glycolide. This family of polymers is more fully disclosed in U.S. Pat. No. 4,994,074, issued Feb. 19, 1991, assigned to Ethicon Inc., which is hereby incorporated herein by reference as if set forth in its entirely. Most preferred are copolymers comprising about 90 mole percent epsilon-caprolactone (PCL) and about 10 mole percent glycolide (PGA) (90/10 PCGA).

Bone grafts of some embodiments of the invention can also comprise biocompatible, biodegradable inorganic fillers in order to provide reinforced implantable medical devices comprising a lubricated surface according to some embodiments of the invention. Such fillers can be fine powders of ceramics comprising mono-, di-, tri-, α-tri-, β-tri-, and tetra-calcium phosphate (TCP), hydroxyapatite, fluoroapatites, calcium sulfates, calcium fluorides, calcium oxides, calcium carbonates, magnesium calcium phosphates, bioglasses, or mixtures thereof.

The biodegradable compositions used to prepare bone grafts of some embodiments of the invention can be used as a pharmaceutical carrier in a drug delivery matrix, or as a cell-based carrier in a tissue engineering application. To form the matrix, an effective amount of therapeutic agent can be added to the polymer or wax prior to, or during, the time of blending. The variety of different therapeutic agents that can be used in conjunction with some embodiments of the invention is vast. In general, bioactive agents which may be administered via pharmaceutical compositions of some embodiments of the invention include, without limitation, antiinfectives, such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators, including general coronary, peripheral and cerebral; central nervous system stimulants; cough and cold preparations, including decongestants; hormones, such as estradiol and other steroids, including corticosteroids; hypnotics; immunosuppressives such as rapamycin; muscle relaxants; parasympatholytics; psychostimulants; sedatives; tranquilizers; naturally derived or genetically engineered proteins, growth factors, polysaccharides, glycoproteins, or lipoproteins; oligonucleotides, antibodies, antigens, cholinergics, chemotherapeutics, hemostatics, clot dissolving agents, radioactive agents and cystostatics.

Scaffold is a porous structural device that allows living tissues to grow into it. A scaffold can form a base which serves as a guide for tissue growth. One approach to repair bone damages is referred to as tissue engineering wherein cells on matrices are used to affect bone repair that would not occur without such an intervention. Bone scaffolds are known in the art, see for example U.S. Pat. Nos. 7,241,486; 7,078,232 and PCT and U.S. Patent Applications WO2006089359, WO2006124937, WO2006095154, US2005/0158535, and US2005/0113934. For example, VITOSS® Scaffold can be used as a bone graft material in the method of some embodiments of the invention. Example 2 further show the use of tissue engineered scaffolds.

Alternative bone graft materials are demineralized bone matrix (DBM) implants that have been reported to be particularly useful (see, for example, U.S. Pat. Nos. 4,394,370; 4,440,750; 4,485,097; 4,678,470; and 4,743,259; Mulliken et al., Calcif Tissue Int. 33:71, 1981; Neigel et al., Opthal. Plast. Reconstr. Surg. 12:108, 1996; Whiteman et al., J. Hand. Surg. 18B:487, 1993; Xiaobo et al., Clin. Orthop. 293:360, 1993; each of which is incorporated herein by reference). Demineralized bone matrix is typically derived from cadavers. The bone is removed aseptically and/or treated to kill any infectious agents. The bone is then particulated by milling or grinding and then the mineral component is extracted (e.g., by soaking the bone in an acidic solution). The remaining matrix is malleable and can be further processed and/or formed and shaped for implantation into a particular site in the recipient. Demineralized bone prepared in this manner contains a variety of components including proteins, glycoproteins, growth factors, and proteoglycans. Following implantation, the presence of DBM induces cellular recruitment to the site of implantation. The recruited cells can eventually differentiate into bone forming cells according to the methods of some embodiments of the invention. Such recruitment of cells leads to an increase in the rate of bone healing and, therefore, to a faster recovery for the subject with massive bone defect.

### Administering Parathyroid Hormone

A parathyroid hormone, its related peptide, or derivatives can typically be administered parenterally, preferably by subcutaneous injection, by methods and in formulations well known in the art. Stabilized formulations of human PTH(1-34) that can advantageously be employed in the present method are described in U.S. patent application Ser. No. 60/069,075, incorporated heroin by reference. This disclosure also contemplates the use of numerous other formulations for storage and administration of parathyroid hormone. A stabilized solution of a parathyroid hormone can include a stabilizing agent, a buffering agent, a preservative, and the like.

The stabilizing agent incorporated into the solution or composition includes a polyol which includes a saccharide, preferably a monosaccharide or disaccharide, e.g., glucose, trehalose, raffinose, or sucrose; a sugar alcohol such as, for example, mannitol, sorbitol or inositol, and a polyhydric alcohol such as glycerine or propylene glycol or mixtures thereof. A preferred polyol is mannitol or propylene glycol. The concentration of polyol may range from about 1 to about 20 wt-%, preferably about 3 to 10 wt-% of the total solution.

The buffering agent employed in the solution or composition of some embodiments of the invention may be any acid or salt combination which is pharmaceutically acceptable and capable of maintaining the aqueous solution at a pH range of 3 to 7, preferably 3-6. Useful buffering systems are, for example, acetate, tartrate or citrate sources. Preferred buffer systems are acetate or tartrate sources, most preferred is an acetate source. The concentration of buffer may be in the range of about 2 mM to about 500 mM, preferably about 2 mM to 100 mM.

The stabilized solution or composition of some embodiments of the invention may also include a parenterally acceptable preservative. Such preservatives include, for example, cresols, benzyl alcohol, phenol, benzalkonium chloride, benzethonium chloride, chlorobutanol, phenylethyl alcohol, methyl paraben, propyl paraben, thimerosal and phenylmercuric nitrate and acetate. A preferred preservative is m-cresol or benzyl alcohol; most preferred is m-cresol. The amount of preservative employed may range from about 0.1 to about 2 wt-%, preferably about 0.3 to about 1.0 wt-% of the total solution.

Thus, the stabilized teriparatide solution can contain mannitol, acetate and m-cresol with a predicted shelf-life of over 15 months at 5° C.

The parathyroid hormone compositions can, if desired, be provided in a powder form containing not more than 2% water by weight, that results from the freeze-drying of a sterile, aqueous hormone solution prepared by mixing the selected parathyroid hormone, a buffering agent and a stabilizing agent as above described. Especially useful as a buffering agent when preparing lyophilized powders is a tartrate source. Particularly useful stabilizing agents include glycine, sucrose, trehalose and raffinose.

In addition, parathyroid hormone can be formulated with typical buffers and excipients employed in the art to stabilize and solubilize proteins for parenteral administration. Art recognized pharmaceutical carriers and their formulations are described in Martin, “Remington's Pharmaceutical Sciences,” 15th Ed.; Mack Publishing Co., Easton (1975). A parathyroid hormone can also be delivered via the lungs, mouth, nose, by suppository, or by oral formulations.

The parathyroid hormone, its related peptides or derivatives are formulated for administering a dose effective for increasing toughness and/or stiffness or brittleness of one or more of a subject's reconstructed bones and/or for reducing the likelihood and/or severity of bone fracture at the site of graft bone union. Preferably, an effective dose provides an improvement in callus bone volume, bone mineral content, cortical bone structure, mass, and/or strength. Preferably, an effective dose reduces the incidence of bone fracture, reduces the incidence of multiple bone fractures, reduces the severity of bone fracture, and/or reduces the incidence of bone fracture at the site where the bone is reconstructed by bone grafts. Preferably, a subject receiving parathyroid hormone, its related peptides or derivatives, also receives effective doses of calcium and vitamin D, which can enhance the effects of the hormone. An effective dose of parathyroid hormone is typically greater than about 0.1 mg/kg/day although, particularly in humans, it can be as large at about 0.4 mg to about 1 mg/kg/day, or larger as is effective to achieve increased toughness, stiffness or brittleness by increasing the callus bone volume or bone mineral content, particularly in cortical bone covering the bone graft, or as is effective to reduce the incidence of fracture at the site of a bone graft. A subject suffering from hypoparathyroidism can require additional or higher doses of a parathyroid hormone; such a subject also requires replacement therapy with the hormone. Doses required for replacement therapy in hypoparathyroidism are known in the art. In certain instances, relevant effects of PTH can be observed at doses less than about 0.4 mg/kg/day, or even less than about 0.1 mg/kg/day.

As shown in the Examples, a mouse model of bone reconstruction is used. Therapeutically effective dosages achieved in one animal model can be converted for use in another animal, including humans, using conversion factors known in the art (see e.g., Freireich et al., Cancer Chemother. Reports 50:219-244 (1996)), Schein et al., Clin. Pharmacol. Ther. 11:3-40 (1970), and Table 1 below for equivalent surface area dosage factors.

TABLE 1

| | From: | | | | |
|---|---|---|---|---|---|
| | Mouse | Rat | Monkey | Dog | Human |
| To: | (20 g) | (150 g) | (3.5 Kg) | (8 Kg) | (60 Kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The hormone can be administered regularly (e.g., once or more each day or week), intermittently (e.g., irregularly during a day or week), or cyclically (e.g., regularly for a period of days or weeks followed by a period without administration). Preferably PTH is administered intermittently once daily for 1-5 days per week for a period ranging from 1 week for up to 20 weeks in patients with massive bone defects. The length of PTH intermittent administration can vary from 1 to 20 weeks, preferably 1 to 15 weeks, still preferably 1 to 10 weeks, more preferably 1 to 6 weeks, still more preferably 2-4 weeks. Preferably, intermittent administration includes administering a parathyroid hormone from 1 week before the bone reconstructive procedure up to 10 weeks following the procedure. Another preferred regime of intermittent administration includes administering the parathyroid hormone for at least about 3 weeks before the bone reconstructive procedure up to 6 weeks following the procedure. Typically, the benefits of administration of a parathyroid hormone persist after a period of administration. The benefits of several weeks of administration can persist for a few months, or more, without additional administration.

Some embodiments of the invention also encompass a kit for enhanced bone-graft healing to be used with some methods of the invention. The kit can contain a vial which contains a formulation of PTH and suitable carriers, either dried or in liquid form and pre-made bone grafts or materials needed for preparing bone grafts including tissue engineered biomatrials/scaffolds, allografts, demineralized bone matrices. The kit further includes instructions in the form of a label on the vial and/or in the form of an insert included in a box in which the vial is packaged, for the use and administration of the compounds and materials. The instructions can also be printed on the box in which the vial is packaged. The instructions contain information such as sufficient dosage and administration information so as to allow a worker in the field to administer the drug. The instructions can further describe hot the bone grafts are prepared or used in a surgical procedure. It is anticipated that a worker in the field encompasses any doctor, nurse, or technician who might administer the drug and/or implant the bone graft.

The examples which follow are illustrative of the invention and are not intended to be limiting.

### Example 1 Intermittent Systemic PTH Treatment Enhances Bone Allograft Healing

This example demonstrates significant anabolic effect of PTH on structural bone allografts, showing substantial improvements in the amount of callus bone formation and graft incorporation resulting in improved union between the host and graft.

### Materials and Methods:

### Surgery:

Femoral allograft surgeries were performed on C57B1/6 mice as previously described [3]. Briefly, a 4 mm mid-diaphyseal segment was removed by osteotomizing the femur and a processed allograft was implanted and secured in place with a stainless steel intramedullary (IM) pin. After 1 week, 8 mice were given daily subcutaneously injections of 0.4 mg/kg PTH (1-34) (Forteo, Eli Lilly and Company, Indianapolis, Ind.) while 5 mice with grafts were given the same volume of saline daily, serving as controls. Animals were sacrificed 6 weeks after surgery, the grafted femurs were carefully dissected and cleaned without disrupting the callus, and the IM pin was carefully removed. The femurs were then stored frozen at −80° C. until evaluation.

### Imaging:

Specimens were scanned at 10.5 micron isotropic resolution using a Scanco VivaCT 40 (Scanco Medical AG, Bassersdorf, Switzerland). Callus bone volume adjacent to the graft was manually segmented from all axial slices that contained grafted bone. The mineralized callus volume, callus bone mineral density (BMD) and total mineral content (BMC) were determined.

### Mechanical Testing—Torsion:

After imaging, the ends of the femurs were cemented into 6.35 mm square aluminum tubes using PMMA in a custom jig to ensure axial alignment and maintain a gage length of 7.3±0.8 mm. Samples were then mounted on an EnduraTec TestBench™ system with a 200 N·mm torque cell (Bose Corp., Minnetonka, Minn.) and tested in torsion at a rate of 1′/sec until failure to determine the torsional stiffness, ultimate torque, ultimate rotation, and strain energy to failure. Mode of Failure was determined by x-ray after testing. As described previously (Reynolds D G, et al; J. Biomech. 2007; E-pub May 22) the mode of failure can be categorized by location of the failure. “Pre-union” failures occur at the graft-host junction resulting in an intact graft that simply pulled out from the callus. “Early union” failures occur near the graft-host junction, but involve partial fragmentation of the graft or host bone. “Mature union” failures occur away from the graft-host junction, indicating that the junction is no longer a point of weakness.

### Statistics:

Student t-tests were performed to distinguish significant results.

### Results:

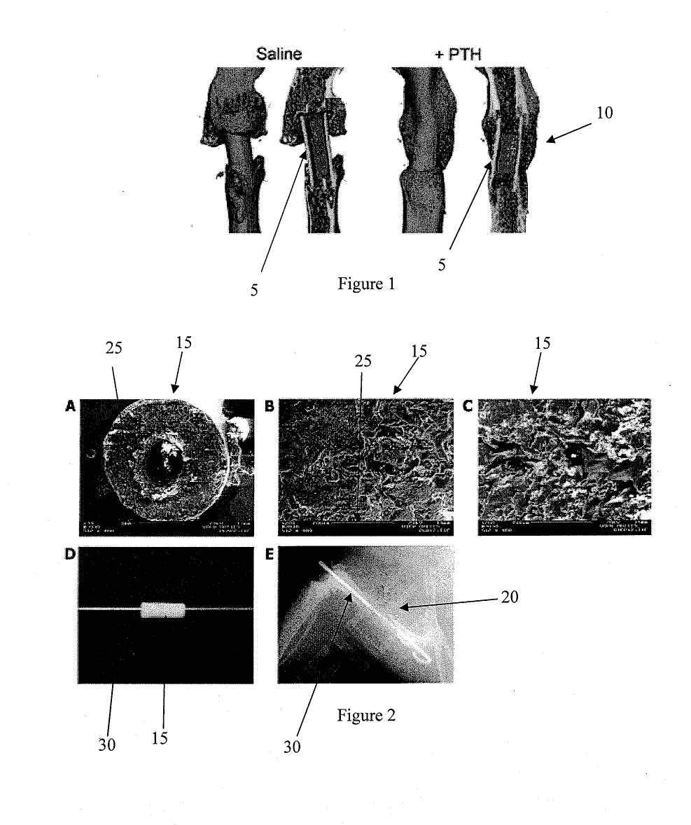

Allografts from animals treated with PTH showed a significant 1.9 fold increase in callus bone volume, 13% reduction in callus BMD but net increase of 60% in total callus mineral content (Table 2). 20% of the saline treated samples had callus bridging the graft from host to host, while 63% of PTH treated samples had bridging callus. As shown in FIG. 1, a longitudinal section from the same specimen is displayed with graft 5. The right panel in this figure shows that PTH enhanced callus 10 volume leading to complete bridging along the length of the graft as compared to saline controls. PTH also enhanced the integration of the host callus onto the graft surface indicating improved union formation.

TABLE 2
Bone volume and mineral quantification by
microCT and biomechanical testing results.

| | | |
|---|---|---|
| Saline | PTH | P-value |

Micro-CT data

| | | | |
|---|---|---|---|
| Callus Bone Volume (mm³) | 2.19 (0.67) | 4.17 (0.80) | 0.001 |
| Callus BMD (mg HA/cm³) | 764 (33) | 665 (17) | 0.001 |
| Callus BMC (mg HA) | 1.69 (0.56) | 2.77 (0.53) | 0.008 |

Torsion Data

| | | | |
|---|---|---|---|
| Ultimate Torque (N*mm) | 8.2 (2.6) | 13.5 (4.7) | 0.02 |
| Torsional Rigidity (N/mm²) | 347 (331) | 1129 (197) | 0.003 |
| Normalized Rotation at T_{Ult} | 3.56 (1.95) | 0.97 (0.23) | 0.04 |
| (deg/mm gage length) | | | |
| Energy to failure | 0.28 (0.13) | 0.25 (0.31) | 0.83 |

Mean (Standard Deviation).

Grafted femurs treated with PTH had a 1.65 fold enhancement in ultimate torque, were 3.3 times stiffer and more brittle, reaching peak torque at a normalized rotation of 0.97 degrees per mm of gage length, while saline control grafts were more ductile, reaching the failure point at 3.56 deg/mm.

All 5 control allograft specimens failed in a “pre-union” mode of failure, whereas 2 of the PTH treated specimens failed in an “early union” mode and 1 had “mature union” failure indicating that PTH treated allografts were better incorporated with the host bone and achieved some degree of union between the graft and host.

The enhancement in callus volume and mineral content in this example is in agreement with data from the fracture-healing literature (Manabe T, et al; Bone. 2007; 40(6):1475-82.). Furthermore, PTH treatment resulted led to improved functional outcome as the PTH-treated allografts achieved greater strength and stiffness approaching 67% and 120%, respectively, of normal unoperated femur properties after only 6 weeks of healing. While previous studies have shown that processed allografts can be revitalized by coating the grafts with rAAV vectors for gene delivery (Ito H, et al; Nature Med 2005; 11, 291-7; Koefoed M, et al; Mol Ther 2005; 12, 212-8), this study suggested that a simple systemic PTH treatment might be sufficient to overcome the limitations of allograft incorporation. Future studies using histology will examine the extent of graft remodeling and “revitalization”. The current findings can have significant implications on the management of human patients receiving allografts and show that systemic PTH treatment can enhance the longevity of structural bone allografts.

### Example 2

### Intermittent, Systemic PTH Treatment Augments Tissue Engineered Reconstruction of Critical Femoral Defects

This example shows that PTH treatment increased bone regeneration and increased the volume of the mineralized callus regardless of the scaffold type used.

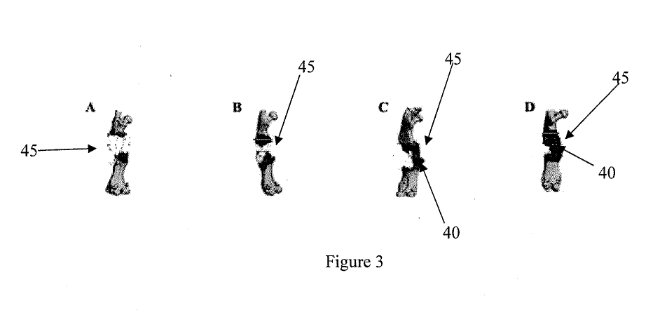

As shown in FIG. 2, an 85:15 PLA/βTCP (PLA) scaffold 15 (panel A; scale bar 100:0 represents 1 mm) was used for bone defect reconstruction. High power SEM images of the scaffold 15 are shown in FIG. 2, panels B and C. (scale bars 25 in B & C represent 200 microns. Arrow head in panel C points to βTCP particles). Titanium pins 30 were passed through the lumen of the scaffolds 15 (panel D) to be used for fixation of the scaffolds when implanted as standalone femoral graft substitutes in critical 4 mm femoral defects in our previously established mouse model (see Example 1). The grafted animals were either treated with daily (5 days/week) injections of PTH or left untreated (Controls). Radiographic image of a PLA scaffold-grafted femur 15, 20 on day 0 is shown FIG. 2, panel E.

A micro-CT rendering of the effect of scaffold type and PTH treatment on bone regeneration in control and PTH-treated animals 9 weeks post-reconstruction showed that 30% of the PTH treated animals developed a mineralized callus 40 that bridged the defect 45 for both scaffolds resulting in union. See FIG. 3, panels C (PLA scaffold) and D (PLA/βTCP scaffold). In contrast, none of the scaffolds in the non-treated controls developed a bridging union. FIG. 3, panels A (PLA scaffold) and B (PLA/βTCP scaffold).

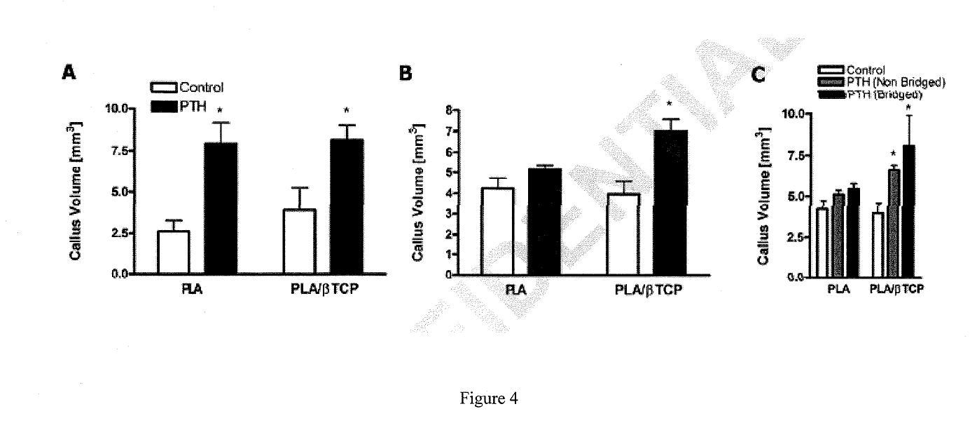

Quantitative analysis of callus volume in PLA and PLA/βTCP scaffolds 6 weeks and 9 weeks post-reconstruction showed that PTH treatment increased the volume of the mineralized callus regardless of the scaffold type. See FIG. 4. Panel A shows a comparison of mineralized callus volume in PLA and PLA/βTCP scaffolds in control and PTH treated animals 6 weeks after bone reconstruction. Panel B compares same 9 weeks after bone reconstruction. Panel C compares the callus volume of specimens that developed a bridging union compared to non-union control and PTH-treated specimens.

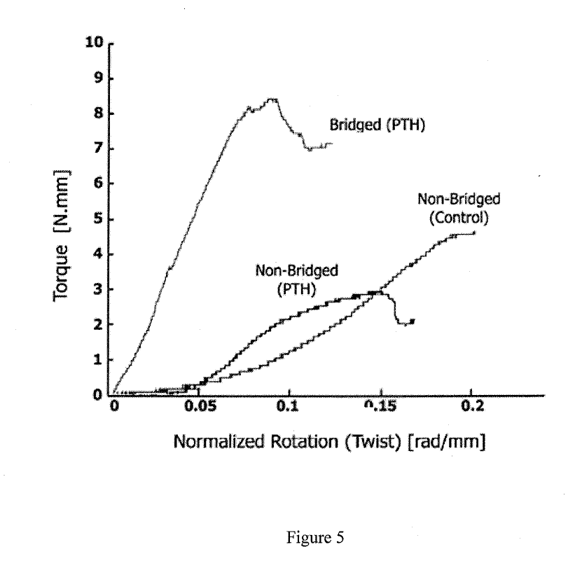

Biomechanical analysis on bridged grafts in PTH treated animals demonstrated a prototypical brittle bone torsion behavior in this bridged specimens. See FIG. 5. When grafts were harvested at 9 weeks and following micro-CT imaging, scaffold-grafted femurs were tested in torsion at a rate of 1°/sec. The representative torque-normalized rotation curve shown demonstrates that PTH treated samples that developed a bridging union exhibit a torsion behavior characteristic of bone with clearly defined linear region, yield and ultimate failure torques, and a relatively brittle fracture. In contrast, non union specimens (control and PTH treated) for the most part did not have defined failure points (up to 80 degrees of rotation) and were quite ductile.

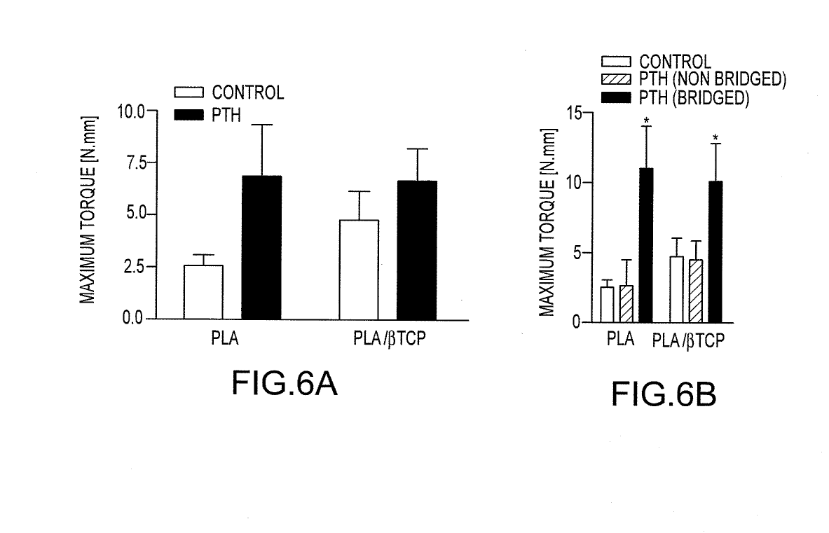

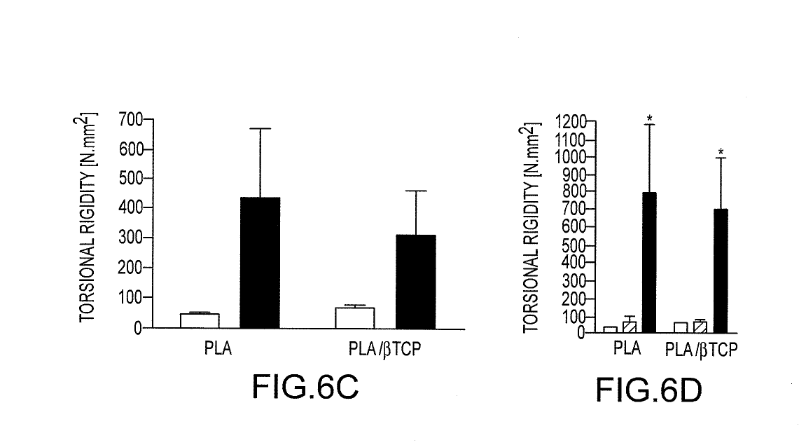

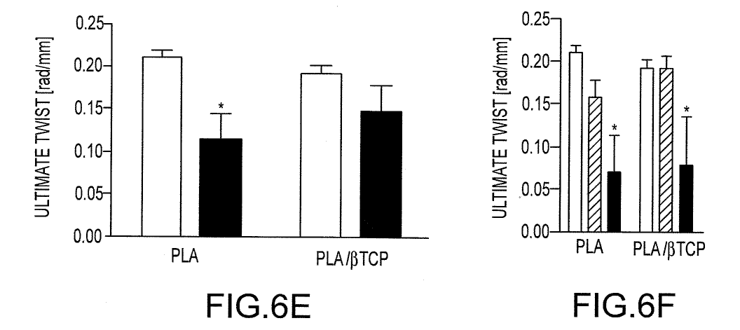

Further biomechanical analysis showed that PTH treatment improved the biomechanical properties of the scaffold-grafted femurs especially in femurs with bridging unions. See FIG. 6. The scaffold-grafted femurs were tested in torsion to determine their biomechanical properties, including maximum torque (panels A&B), torsional rigidity (panels C&D), and ultimate normalized rotation or twist (panels E&F). Panels A, C, and E show the average properties for both scaffold types in control and PTH treated animals. Panels B, D, and F show the maximum torque, torsional rigidity, and ultimate twist, respectively, of specimens that developed a bridging union compared to non-union control and PTH-treated specimens. Data are presented as mean+SEM. Asterisk indicates significant differences from control (p<0.05).

A skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

### Example 3

### Micro-CT-Based Measurement of Cortical Bone Graft-to-Host Union

### Materials and Methods:

### Experimental Model

4-mm intercalary defects in C57B1/6 mouse femurs were reconstructed using either the live cortical bone graft from the same mouse (autograft) or a devitalized bone graft from a donor mouse (allograft) and secured in place with an intramedullary pin. Only mice that were sacrificed at 6 weeks (n=7 autografts and 8 allografts) or 9 weeks (n=12 autografts and 7 allografts) after surgery were included in this study. Femurs were disarticulated from the hip and knee joints and the intramedullary, stainless-steel pins were removed carefully. Specimens were moistened with saline and frozen at −20° C. until thawed for micro-CT imaging and subsequent biomechanical testing.

Specimens were scanned at 13.9 micron resolution using the Explore Locus SP scanner (GE Healthcare Technologies, London, ON) at 80 kVp and 80 mA with 415 projections of 1700 ms integration time. GE MicroView software was used for measuring bone volume and cross-sectional organization. To compensate for slight variations in the scanner, a threshold was determined for each scan using a standardized automatic threshold-selection feature of the GE MicroView software that utilizes the Otsu method. This determines the threshold which maximizes the variance between the groups of pixels. The selected threshold was consistently verified against the user's perception of the boundary of the mineralized bone. Manual segmentation of the graft and callus bone volumes (BV_{Graft}, BV_{Callus}) was performed on axial cross sections of the grayscale images as previously described. The cross-sectional polar moments of inertia (PMI) were computed for each slice throughout the grafted region. The maximum, minimum, and average PMI (PMI_{Max}, PMI_{Min}, PMI_{Ave}) were recorded for each specimen. In circular, prismatic shafts, the PMI correlates directly with torsional rigidity and inversely with the shear stress. The torsional biomechanical properties of the grafted femurs were then determined using the EnduraTec TestBench™ system (200 N·mm torque cell; Bose Corporation, Minnetonka, Minn.) at a rate of 1°/sec. Raw data from the testing was plotted as torque vs. rotation (normalized to the measured gage-length) and used to determine the ultimate torque and torsional rigidity for each specimen. The torsional rigidity was determined as the maximum slope of the curve between the start of the test and the maximum torque. Specifically, we used a sliding window ⅕^{th} the width of this region to determine the maximum slope.

### Union Ratio Algorithm

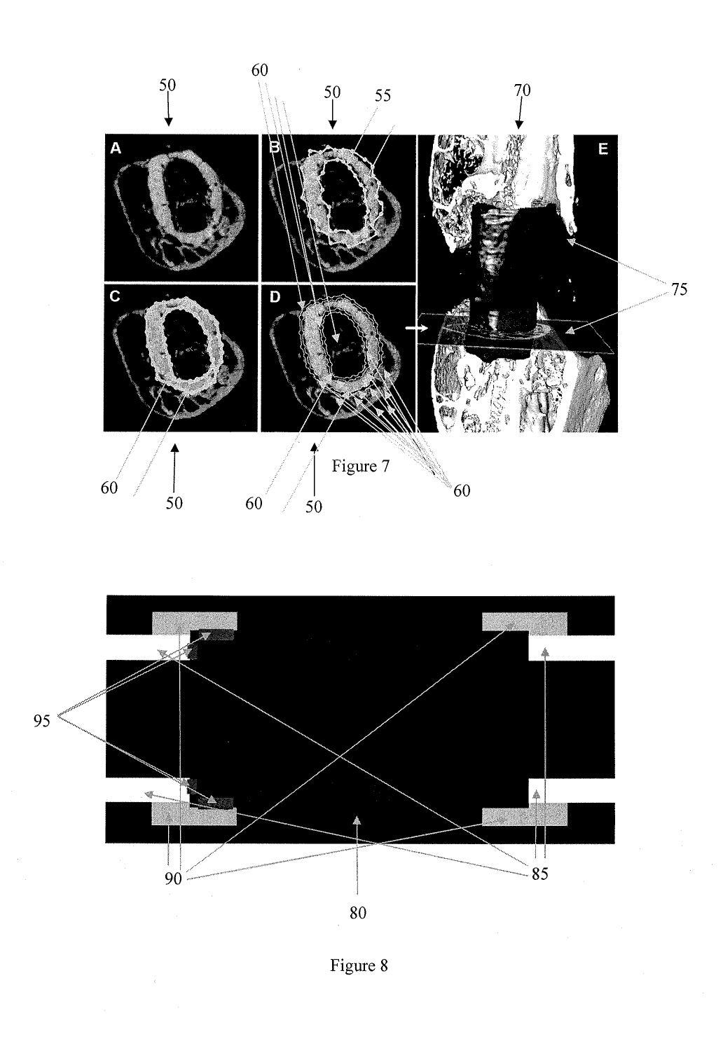

FIG. 7 illustrates an exemplary application of an embodiment of a Union Ratio algorithm. A user outlines the surface of the graft using contours on transverse micro-CT slices 50 (FIG. 7A). The semi-automated algorithm developed using MATLAB then optimizes the manually defined contours drawn around the endosteal and periosteal surfaces (FIG. 7B). The contours are first snapped to the graft boundary by edge-detection 60 (FIG. 7C), then dilated into darker regions away from the graft surface, finding gaps 65 between graft and callus, if any exist (FIG. 7D). The resulting 2D contour from one slice is then copied to the next slice and the edge detection and gap-finding operations are performed. This process is repeated on each slice until the entire graft is enclosed in contours. A smoothed 3D shell 70 is generated from the contours using MATLAB's isosurface function (FIG. 7E). The footprint of bone penetrating the shell therefore defines connection areas 75 between the graft and host or callus. Summed over the entire surface area of either half of the graft shell, the lesser area of the connections normalized by the total surface area for either proximal or distal half is defined as the Union Ratio.

Custom software was written in MATLAB (The Mathworks, Natick, Mass.) for the analysis of the Union Ratio from the micro-CT images. An active contouring algorithm was adapted for the semi-automated generation of a shell around the graft. First, contours are drawn around the periosteal and endosteal surfaces of the bone graft in a single transverse micro-CT slice which has been lightly low-pass filtered using a 2D Gaussian filter (σ=1.8 pixels; FIGS. 7A and 7B). The contour then snaps to the edge of the graft based on the 2D gradient of the grayscale image using a Prewitt filter (FIG. 7C). Lastly, the contour dilates to a neighboring pixel with the darkest grayscale intensity along a 4 pixel-long line drawn from each contour point perpendicular to the line that connects the two contour points on each side of the current contour point. Thus the contour dilates into the void space between the graft and callus bone if it exists (FIG. 7D). Because the contour snaps to the gradient between contrasting pixels, the contour point will shift to the material of lesser radiopacity. Generally, this means it shifts off the dense cortical graft, onto either newly mineralized callus (woven bone is less radiopaque than organized lamellar bone), or onto unmineralized soft tissue adjacent to the graft. Cubic spline interpolation was used to smoothly join the contours. The contour from the previous slice is then copied onto the next where the edge-detection and void space search processes are repeated under operator supervision and modification, until the entire length of the graft is contoured to create a shell around the graft. The shell is then meshed using triangular elements that are used to quantify the amount of graft area in contact with host bone or mineralized callus by summing 1/3 of the area of each triangle element for each vertex that falls within a voxel with a grayscale value greater than the threshold used to define mineralized tissue. The proximal and distal halves of the graft are evaluated separately, and the lesser ratio of the connected surface area to total graft surface area is used in the analysis and assigned as the value of the Union Ratio, to account for any variation in graft size in our standardized model.

### Algorithm Validation

FIG. 8 illustrates an idealized cylindrical graft 80 between host cortical bone 85 and callus 90 was digitally generated in MATLAB and used to validate the Union Ratio measurement. The graft was given defined rectangular regions of union to the host directly as well as between the graft and the callus forming around it. The theoretical union area 95 based on the idealized geometry projected onto the curved surface was 2173.2 pixels². Using the contouring computational algorithm, the measured area was 2171.4 pixels² resulting in a measurement error of only 0.08%.

A digital model with standard hollow cylindrical geometry was created in order to validate the calculations used to measure the connected surface area. This model was generated as an idealized graft between two host ends, with geometrically-defined connections simulating callus originating from the host tissue. The hollow cylindrical model was generated with thickness of 15 pixels and outer diameter of 50 pixels, yielding a relative resolution similar to the resolution of the real micro-CT images (typical allograft cortical thickness was 180-200 microns (13-14 pixels) thick, and about 1.25-1.55 mm (90-110 pixels) in diameter. Predetermined areas of connectivity were created directly between the graft and host as rectangular prisms that either attached to the end surface of the graft or intersected the periosteal surface of the graft connecting it to the callus. This idealized model was then contoured and the Union Area was computed as described for the experimental grafts.

### Statistical Analysis

Comparisons of autograft and allograft Union Ratio data at the different time points were performed using 2-way analysis of variance and Bonferroni post hoc multiple comparisons.

To evaluate intraoperator and interoperator error in the estimation of the Union Ratio, a subset of 2 specimens from each group (8 specimens total) was randomly selected to be repeated by the first operator (DGR) as well as performed and repeated by another trained operator (MOP). The average percent error between measurements was calculated by the absolute value of the difference between measures divided by the average measurement. As described by Lodder et al (12) the coefficient of variation (CV) is the standard deviation between measurements normalized by the mean of the paired measurements, calculated as

$$\% CV = \frac{\sqrt{\sum {( a - b )}^{2} / 2 n}}{( {M}_{a} + {M}_{b} ) / 2} \times 100$$

where a and b are the first and the second measurements, Mₐ and M_{b} are the mean values for the two groups, and n is the number of paired observations. Intraclass correlation coefficients (ICC) to evaluate the concordance, or agreement, between measurements within and between operators were computed. This is defined as the difference between the overall variation and the measurement variation, divided by the sum of the measurement and overall variation. The ICC ranges between 0 and 1 where 1 is perfect concordance.

### Results:

### Algorithm Validation

To validate the semi-automated contouring algorithm and computation of the Union Ratio, we created a digital model that resembles a graft connected to host bone and callus by a footprint of defined dimensions. The predetermined connected area which accounts for the curvature of the cylindrical model surface was computed to be 2173.2 pixels2. Using the contouring method and the MATLAB algorithm, the Union Area was determined to be 2171.4 pixels2, resulting in a measurement error of only 0.08%.

### Union Ratio of Autografts and Allografts

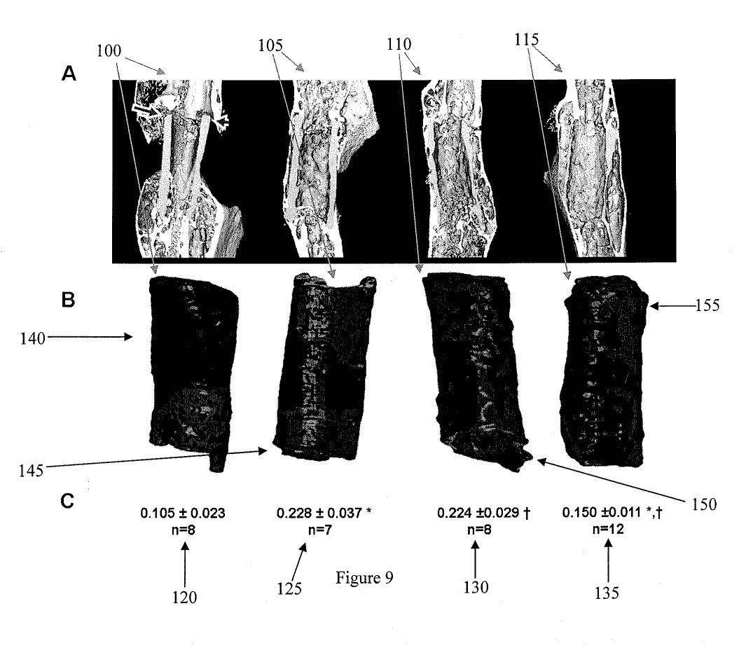

FIG. 9 illustrates representative micro-CT sagittal sections of a 6 week allograft 100, a 6 week autograph 105, a 9 week allograft 110, and a 9 week autograft 115; and corresponding union area maps for the 6 week allograft 140, the 6 week autograft 145, the 9 week allograft 150, and the 9 week autograft 155; and Union Ratio numerical values for the 6 week allograft 120, the 6 week autograft 125, the 9 week allograft 130, and the 9 week autograft 135. In union area maps 140, 145, 150, and 155 grey shading indicates areas where the graft is connected to the host. In determining the Union Ratio, the proximal and distal halves of the graft were evaluated separately, and the lowest value of the union area normalized by the surface area was reported as the Union Ratio (C; mean±SEM). Significantly different means are labeled t for p<0.05 between time points for each graft type and * for p<0.05 between graft types at each time point illustrates the typical differences in the union with host bone and callus between allografts and autografts at 6 and 9 weeks. At 6 weeks, the Union Ratio of autografts was nearly double that of allografts (p<0.05). The areas of union were also more uniformly distributed along the length of the autografts compared to the allografts for which new bone formation was restricted to the host bone at the ends of the grafts (FIG. 3). At 9 weeks, the allografts' Union Ratio was 2.2 times that of 6 week allografts (p<0.05), while the autografts' Union Ratio declined 33% from 6 to 9 weeks (p<0.05).

We also investigated the intra- and inter-operator sources of error in the measurement of the Union Ratio. The average percent error between operators' measurements was 12% and the coefficient of variation (CV) was 9.7%. The intra-operator ICC was 0.930 for DGR and 0.949 for MOP while ICC between different operators (DGR and MOP) was 0.926. These results indicate that the measurements were remarkably reproducible.

Correlations between Union Ratio and Torsional Properties

To estimate the effects of the Union Ratio on the torsional biomechanical properties, we performed univariate linear regression analyses. When autografts and allografts at all time points were grouped, the regression analysis identified weak, yet significant, associations between the Union Ratio and the torsional properties (Table 3).

TABLE 3
Coefficients of determination (R²) and p-values for
univariate linear regression of graft Union Ratio
with ultimate torque and torsional rigidity.

| | Ultimate Torque | | Torsional Rigidity | |
|---|---|---|---|---|
| Group | R² | ^{§}P | R² | P |
| Autografts & Allografts | 0.12 | <0.04 | 0.15 | <0.02 |
| Autografts | 0.15^{†} | N.S. | 0.05^{†} | N.S. |
| Allografts | 0.58 | <0.001 | 0.51 | <0.003 |

^{†}indicates inverse linear correlations (i.e. negative slope).
^{§}P values for the two-sided test of the null hypothesis that the slope of the regression line is zero.
N.S. indicates p > 0.05.

However, when analyzing the allograft data separately the correlation was much stronger. By contrast, there were no significant associations between the autografts' Union Ratio and torsional properties. Taken together, these results suggest that the Union Ratio is a significant indicator of functional strength in the devitalized allografts that undergo no or little remodeling over the first 9 weeks of healing, while it does not correlate with the biomechanical properties of autografts that undergo a robust remodeling (6) such that the Union Ratio actually decreases Between 6 and 9 weeks due to excessive graft resorption.

To account for other variables that contribute to the biomechanical properties of the grafts, we investigated multivariate correlations between micro-CT parameters and torsional properties as previously described. When included as an independent variable, the Union Ratio was a significant, predictive variable that increased the regression coefficients for rigidity and strength of 6 and 9 week autografts and allografts as a group.

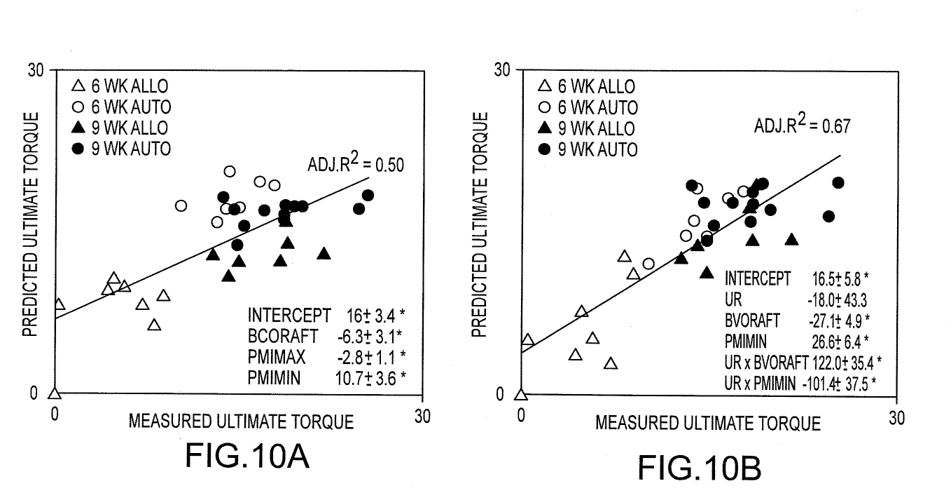

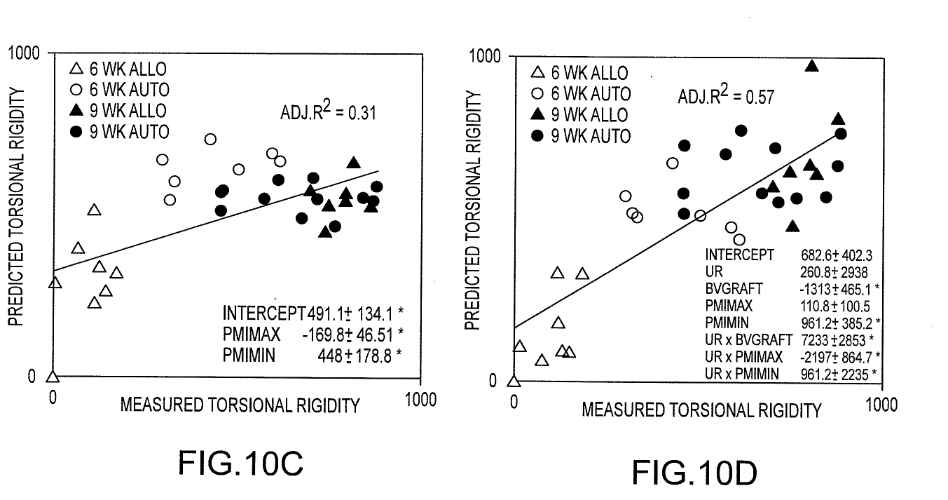

To determine the Union Ratio's ability to improve the correlation between structural measures and mechanics, multivariable regression was performed twice, once without the Union Ratio, and again with the Union Ratio as an independent and interacting term. A regression analysis was performed without Union Ratio (A and C), and with Union Ratio (B and D) for the combined set of autografts (Auto) and allografts (Allo). Adjusted R² and the significant regression coefficients are indicated on each graph with their (±standard error). * indicates that the independent variables or the interaction terms are significant (p<0.05). Without Union Ratio, BV_{Graft} PMI_{Max} and PMI_{Min} were found to correlate with T_{Ult} yielding an adjusted R²=0.50 (FIG. 10A) and PMI_{Max} and PMI_{Min} were found to correlate with TR yielding an adjusted R²=0.31 (FIG. 10C). Including Union Ratio in the regression model improved the correlation coefficients. The ultimate torque correlated significantly with the combination of Union Ratio, BV_{Graft}, PMI_{Min} and the interaction terms Union RatioxBV_{Graft} and Union RatioxPMI_{Min} (adjusted R²=0.67, FIG. 10B). The torsional rigidity correlated significantly with Union Ratio, BV_{Graft}, BV_{Callus}, PMI_{Max}, PMI_{Min}, and the interaction terms Union RatioxPMI_{Max}, and Union RatioxPMI_{Min} (adjusted R²=0.57, FIG. 10D).

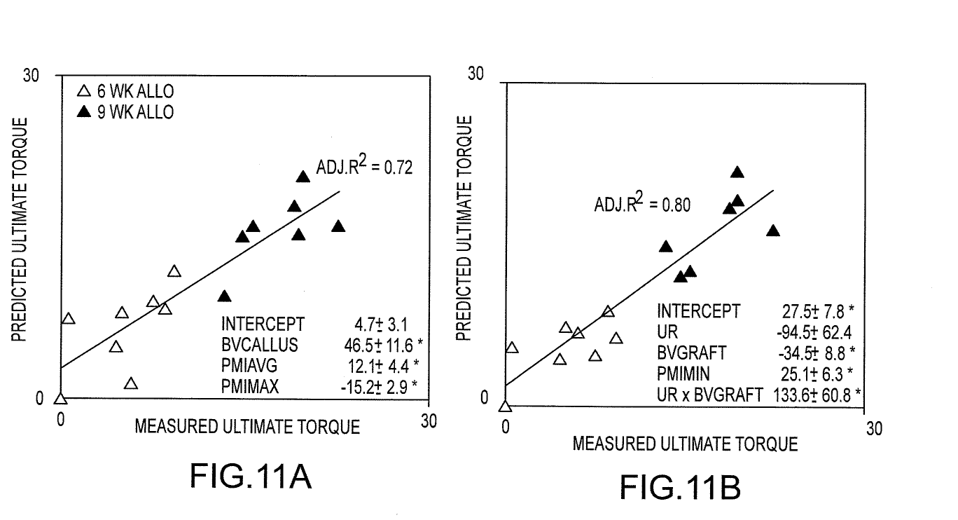

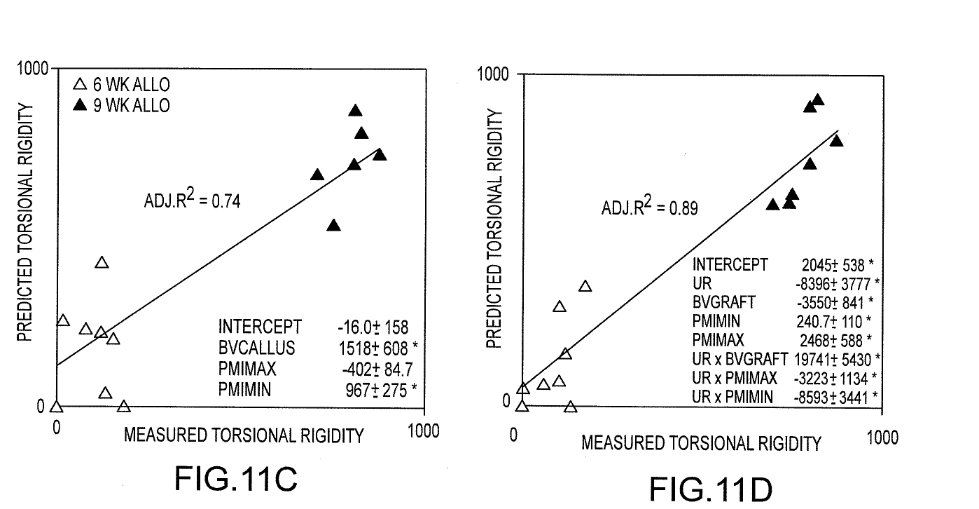

When allografts were analyzed separately without including the Union Ratio in the multivariate regression analysis, BV_{Callus}, PMI_{Ave} and PMI_{Max} correlated with the ultimate torque with an Adjusted R²=0.72 (FIG. 11A). When the Union Ratio was included in the model, Union Ratio, PMI_{Min}, BV_{Graft}, and Union RatioxBV_{Graft} correlated with the ultimate torque, increasing the adjusted R² to 0.80 (p<0.05) (FIG. 11B). Likewise, the correlation with the torsional rigidity of allografts significantly improved with the addition of the Union Ratio from an adjusted R² from 0.74 to 0.89 (p<0.05) with the combination of the Union Ratio, BV_{Graft}, PMI_{Max}, PMI_{Min} and the interaction terms with the Union Ratio: Union RatioxBV_{Graft}, Union RatioxPMI_{Max}, Union RatioxPMI_{Min} (FIGS. 11C & D). The regression analysis was performed without Union Ratio (A and C), and with including Union Ratio (B and D) for allografts only. Adjusted R² and the significant regression coefficients are indicated on each graph with their (±standard error). * indicates that the independent variables or the interaction terms are significant (p<0.05).

### Discussion:

Despite the high incidence of bone fractures and the clinical development of safe and effective anabolic/osteogenic therapies for bone healing (i.e. teriparatide, BMP-2), the lack of a non-invasive outcome measure of biomechanical healing of fractured bone continues to limit our ability to define non-unions and evaluate new therapies for unmet clinical needs. Previously we attempted to correlate established micro-CT parameters with torsional properties in the murine femoral auto and allograft model, and found that we could at best predict 50% of the biomechanical properties of the mouse grafted femurs (6). This poor correlation is largely explained by the fact that none of the established micro CT parameters are not capable of quantifying the extent of cortical bone union between the graft and the host, which intuitively should be directly related to strength of the bone. Therefore, we developed and validated a novel algorithm to quantitatively estimate the union between graft and host bone based on micro-CT data. Our results highlighted the differences in healing due to graft type, as well as the changes in union and osseointegration patterns over time. Furthermore, one-to-one correlations demonstrated that the Union Ratio was a significant predictive variable of the biomechanical properties of the devitalized allografts, but not the live autografts.

Quantifying the Union Ratio of live autografts and devitalized allografts corroborated previously-published qualitative observations regarding the biology and biomechanics of healing in both cases. Histological evidence shows that devitalized allografts induce a foreign body reaction that encases the graft in a fibrous layer initially which can be gradually overcome with progression of the creeping callus from the host bone that typically remains restricted to the graft ends (5). Our results now show that the mitigation of non-union by 9 weeks, when the callus finally penetrates the fibrous capsule and integrates with the devitalized allograft, significantly increases the ultimate torque and torsional rigidity.

In the case of autografts, the Union Ratio did not independently correlate with torsional properties, while the allografts' Union Ratio significantly correlated with the torsional properties (Table 1). We hypothesize that these results reflect fundamental biological differences in the healing of live autografts and the devitalized allografts which arise from the contribution of periosteal cells in live autografts that are absent in devitalized allografts. We have previously shown that autograft repair is facilitated by both endochondral bone formation at the host-graft junction and by intramembranous bone formation along the entire length of the graft as early as 2 weeks post-transplantation, and undergoes significant remodeling by 4 weeks. This results in the formation of a new bone collar that bridges the entire length of the autograft by 4 weeks, which is also apparent in this study at 6 and 9 weeks in FIG. 3. We hypothesize that this new bone collar begins to assume a significant share of the in vivo loading, and therefore the autograft begins to experience significant stress-shielding and undergoes rapid and substantial resorption (by up to 57%) by 6 weeks, thus rendering its contribution to mechanical properties of the femur negligible. Therefore, whether or not the remaining graft has a high degree of union to the new cortical shell plays little role in the overall mechanical strength. In contrast, devitalized allografts completely rely on endochondral bone formation initiated by the host at the host-graft cortical junction, with no evidence of periosteal bone formation along the length of the allograft, and no appreciable graft resorption. The result is significant callus formation that is limited to the host-graft junction and whose union with the allograft is crucial to load transmission and mechanical strength.

Furthermore, our multivariate correlations do not account for the complete cortical bridging observed in 100% of the autografts at 6 and 9 weeks, which likely makes a significant contribution to the biomechanical properties. The development of a measure of this type of union could potentially contribute to the ability to predict the mechanical stability of healing bone autografts.

Previously published studies have attempted to estimate fracture and graft union using histological and stereological techniques and 2D plain radiographs. But those approaches are prone to inaccuracies as they do not account for the 3D nature of the cortical healing. Recent reports have attempted to utilize high-resolution micro-CT imaging to characterize fracture non-union. Those studies defined measures of union based on counting the number of bridged cortices in planar sections or relied upon qualitative 3D rendering of the fracture sites to demonstrate union or the lack thereof in response to the treatment. Therefore, our study not only reports the development of a novel quantitative measure of union, but to the best of our knowledge it is also the first to report direct correlations between the graft and host degree of union and the biomechanical properties of the reconstructed bone, which could have important applications in longitudinal preclinical and clinical studies of bone repair and grafting.

The Union Ratio has significant clinical implications as a novel quantitative biometric which merits further study in large animal pre-clinical using clinical CT scanners. Various preclinical and clinical studies have been performed to treat bone injuries with adjuvant treatments to enhance healing and bone formation around allografts, enhance their incorporation and remodeling, and their biomechanical properties and durability. Such treatments include the use of BMPs and other growth factors, co-engraftment with mesenchymal stem cells, the use of locally administered gene therapy, engineered bone graft substitutes, and recently, the use of the bone anabolic factor such as parathyroid hormone, to name a few. The evaluation of the repair quality and osseointegration in preclinical animal models can be accomplished by destructive biomechanical testing. However, the evaluation of clinical patients has to date been mostly based on non-quantitative radiographic outcomes since destructive biomechanical testing is not an option.

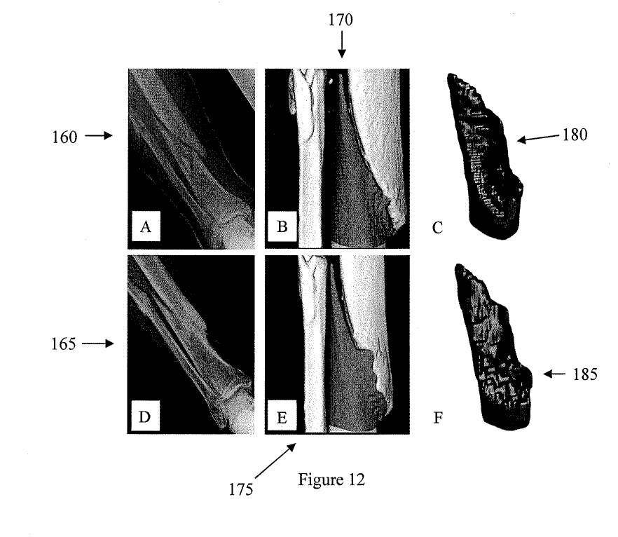

To demonstrate clinical utility of our algorithm on CT scans of clinical resolution, we retrospectively analyzed clinical CT images of an anonymous patient with a prolonged non-union (>4 months) tibial fracture, which was subsequently non-surgically treated with teriparatide. We used our custom MATLAB software to contour the segment of bone on one side of the fracture site similarly to contouring around the murine graft. The surface area forming union to the other side of the fracture was then estimated by the software. After 4 months of treatment, the patient had a 2.8 fold increase in the mineralized Union Area connecting the fractured segments which underscored the functional outcome of the patient being able to finally bear weight on the healing leg. FIG. 12 illustrates clinical x-rays of an anonymous patient's fractured tibia before and after 4 months of teriparatide therapy, 160 and 165, respectively and CT scan data of the patient before and after 4 months of teriparatide therapy, 170 and 175, respectively; and Union Area, shown in grey in 170 and 175, of the patient before and after 4 months of teriparatide therapy, 180 and 185, respectively. The effects of teriparatide on fracture healing were quantified as a 2.8 fold increase in Union Area.

### Example 4

### Teriparatide (PTH 1-34) Treatment Improves Grafted Femur Biomechanics

The objective in this study was to determine whether, in the context of bone allografts, bone graft-to-host union, bone mechanics, bone volume, and mineral content are improved by intermittent systemic PTH treatment at 6 weeks after allograft implantation.

### Materials and Methods:

### Experimental Model

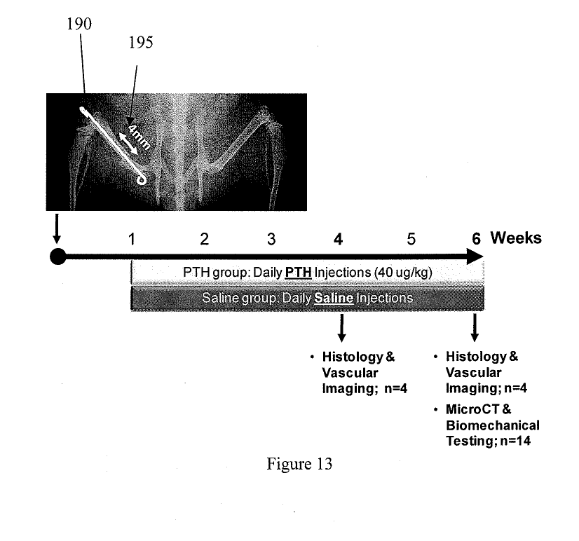

Four-mm long bone allografts were harvested from donor mice, and were processed and implanted into intecalary defects 195 in the femur of other mice and secured in place using a stainless steel intramedullary pin 190, as shown in FIG. 13. One week after surgery, daily injections of 40 μg/kg hrPTH (1-34) (Lilly, Inc., Indianapolis, Ind.) were initiated in half of the mice, while the others received injections of saline control. Weekly x-rays were taken to monitor progression (Faxitron X-Ray LLC, Wheeling, Ill.).

### Biomechanical Study

One cohort of the study groups used 14 mice from each treatment (PTH and control) for imaging and mechanical material testing and were sacrificed 6 weeks after surgery. Each femur was harvested by disarticulating the hip and knee and removing the intramedullary stainless-steel pin. Specimens were moistened with saline and frozen at −20° C. until thawed for micro-CT imaging and torsion testing. Specimens were scanned at 12.5 Mm isotropic resolution using the Scanco VivaCT 40 (Scanco Medical AG, Bassersdorf, Switzerland). From these 3D images, the graft and callus bone volumes (BV_{Graft}, BV_{Callus}) were measured by manual segmentation, followed by standardized thresholding at a grayscale corresponding to 750 mgHA/cm³. The cross-sectional polar moment of inertias (PMI) were computed for each slice throughout the grafted region and the maximum, minimum and average PMI (PMI_{Max}, PMI_{Min}, PMI_{Ave}) were investigated to determine their contribution to the biomechanics of the grafted femurs.

The Union Ratio was also calculated. The Union Ratio measures the graft surface area upon which mineralized callus has formed. If the voxels adjacent to the graft surface are boney callus, the area of that region of the graft is measured and normalized to the total graft surface area. Imaging data (not shown) indicated the bare surface of the graft in blue, with regions of union to the callus depicted as red. Each half (proximal and distal) of the graft was evaluated separately and the lesser ratio of union area to total graft surface area was given as the Union Ratio for that specimen. Callus formation that bridged from host-to-host over the graft was determined by evaluating serial axial cross sections from micro-CT images and given a binary result. These samples were then mechanically tested in torsion. Yield torque (T_{Yield}), ultimate torque (T_{Ult}), torsional rigidity (TR), toughness (or work to failure) and the twist at ultimate torque were determined for each specimen. Finally, the mode of failure for each specimen was determined using an x-ray image analysis (not shown).

### Vascularization and Histological Study

A second cohort of 16 animals underwent the same surgery with sacrifice of 8 animals at 4 weeks and 8 animals at 6 weeks post-surgery to evaluate the degree of vascularization of the graft and callus region using vascular profusion as described previously (Duvall 2004). Half of the animals were treated with PTH, and the other half with saline.

### Vascular Perfusion

On the day of sacrifice, animals were injected with a fatal dose of ketamine and xylazine and their vasculature was perfused using a syringe pump through a needle placed into the left ventricle of the heart. The right atrium was also punctured to allow the blood to drain out. They were first perfused with heparinized (100 units/ml) saline to prevent blood clotting, followed by 10% neutral-buffered formalin, and lastly with lead-chromate contrast agent (Microfil 122, Flow Tech, Inc. Carver, Mass.). Samples were fixed in 10% formalin overnight followed by harvest of the femur and pin extraction. Samples were micro-CT scanned once after fixation, and again after EDTA decalcification. Using both scans the vasculature was evaluated within the mineralized callus. The vessel volume, thickness, spacing and vessel number was determined.

### Histology

After micro-CT imaging for vascular analysis, specimens were processed for histology. Mid-sagittal sections were stained with alcian blue, hematoxylin, eosin and orange G. Micro-CT images were manually resliced using NIH ImageJ software in the same plane as the histology sections to compare the imaging modalities (data not shown).

### Statistical Analysis:

Student t-tests were used to compare differences between PTH treatment and saline treatment for each of the micro-CT imaging measures, and biomechanical testing results.

Univariate regression analysis was used to determine the degree of correlation between micro-CT imaging derived measures and ultimate torque, yield torque and torsional rigidity. Multivariate linear regression analysis was used to determine combinations of micro-CT parameters that correlated with the torsional mechanical properties. Stepwise selection regression analysis was used to optimize the combination of significant (p<0.05) independent variables in a linear model. This was performed using SAS 9.1 (SAS Institute Inc., Cary, N.C.).

### Results:

Bone Analysis from Micro-CT Imaging

Observations from Micro-CT imaging shown (not shown) revealed that in PTH treated specimens host callus formation around the graft were larger and packed with regions of trabecular bone. Intramedullary callus was also present to a greater extent in PTH treated animals. There were also fewer apparent non-unions visible in PTH treated specimens. Bridging over the graft from host-to-host was present in 6 of 14 specimens from saline treated control animals, and 8 of 14 specimens treated with PTH.

At 6 weeks after surgery, PTH treatment significantly increased BV_{Callus} by 93%, with a noteworthy increase in BV_{Intramed} of 217%. The enhanced bone formation resulted in a significant 38% and 26% increase in PMI_{Ave} and PMI_{Max}, respectively. This was predominately due to an increase in cross sectional area due to the increase in bone volume fraction of the callus, and not a change in the outer diameter of the callus—the maximum outer radius was 1.8±0.2 mm for saline controls and 1.7±0.3 mm for PTH treated animals. In PTH treated animals, bone mineral density of the callus was significantly less dense by 14%, but the net callus total mineral content was still significantly greater by 67% because the bone volume fraction within the callus was 52% greater. The graft bone volume was not different between treatment groups at 6 weeks suggesting that there was no increase in graft resorption with PTH treatment. This was verified with histology which revealed no difference in the resorption spaces on the graft surface area. The Union Ratio, a measure of the relative surface area upon which callus bone has formed, was significantly 76% greater (p<0.01) (Table 4).

Sagittal micro-CT images of the proximal graft-host interface correspond with the Hematoxylin/Eosin and Orange G histology images (data not shown) were performed on animals 4 weeks after surgery while and 6 weeks after surgery. 40 μg/kg of PTH(1-34) was administered daily in test groups while the others received saline. Specimens were also used for volumetric vascular analysis by μCT and are thus perfused with lead-chromate contrast agent which appears white on μCT and black in histology. Of note is that cartilaginous callus persisted in 4 week PTH treated specimens, PTH treatment enhanced the ratio of bone-to-hematopoetic marrow within the callus and overcame the fibrous the gap between callus and graft bone, thus forming callus directly on the surface of the graft. PTH treated specimens also showed enhanced intramedullary bone formation.

Bone volume quantification was performed from micro-CT imaging. Quantification of micro-CT results for a control and PTH treated (FIGS. 16E-H) specimen was performed. Cross sectional polar moment of inertia, graft were determined. Callus and intramedullary callus bone volumes were quantified for each specimen in a region of interest that extended from the proximal axial slice containing bone graft through the distal slice. These regions were manually segmented and quantified for bone volume, bone mineral density and bone mineral content at a threshold corresponding to 750 mgHA/cm³. The total depth of penetration of callus into the intramedullary cavity from both ends was also measured. The trabecular-like region within the shell of the exterior callus was segmented for trabecular analysis to quantify BVF, Tb.No., Tb.Th., and Tb.Sp.

TABLE 4
Micro-CT imaging parameters of grafted femurs.

| | Saline | | PTH | | |
|---|---|---|---|---|---|
| PMI_{Min} (mm⁴) | 0.41 | (0.11) | 0.51 | (0.26) | |
| PMI_{Ave} (mm⁴) | 0.84 | (0.14) | 1.16 | (0.32) | ** |
| PMI_{Max} (mm⁴) | 1.72 | (0.59) | 2.17 | (0.46) | * |
| BV_{Graft} (mm³/mm) | 0.84 | (0.066) | 0.82 | (0.067) | |
| BV_{Callus} (mm³/mm) | 0.54 | (0.14) | 1.04 | (0.3) | ** |
| BV_{Intramed} (mm³/mm) | 0.063 | (0.042) | 0.2 | (0.081) | ** |
| Intramed Penetration | 1.73 | (0.57) | 2.22 | (0.71) | |
| Depth (mm) | | | | | |
| BMD_{Callus} (mgHA/cc) | 774 | (28) | 667 | (16) | ** |
| BMC_{Callus} (mgHA) | 1.62 | (0.47) | 2.7 | (0.76) | ** |
| Callus trabecular BVF | 0.379 | (0.21) | 0.576 | (0.046) | * |
| Callus Tb. N. | 6.38 | (1.75) | 13.2 | (1.0) | ** |
| Callus Tb. Th. | 0.0722 | (0.023) | 0.149 | (0.198) | |
| Callus Tb. Sp. | 0.182 | (0.054) | 0.065 | (0.007) | ** |
| Union Ratio | 0.129 | (0.088) | 0.277 | (0.068) | ** |

Mean (SD). n = 14 per treatment.
* p < 0.05,
** p < 0.005

TABLE 5
Micro-CT imaging parameters of intact contralateral femurs.

| | Saline | | PTH | | |
|---|---|---|---|---|---|
| | Contralateral | | Contralateral | | |
| M-L Periosteal Diameter (mm) | 1.76 | (0.03) | 1.85 | (0.08) | |
| A-P Periosteal Diameter (mm) | 1.22 | (0.001) | 1.30 | (0.05) | |
| Cortical Thickness (mm) | 0.180 | (0.006) | 0.199 | (0.007) | * |
| Cortical Bone Density | 1201 | (4) | 1179 | (21) | |
| (mgHA/cc) | | | | | |
| Cross-Sectional Area (mm³) | 0.69 | (0.01) | 0.82 | (0.05) | * |
| Polar Moment of Inertia (mm⁴) | 0.27 | (0.01) | 0.36 | (0.03) | * |

Mean (SD) N = 8 per treatment.
* p < 0.05

### Biomechanical Testing Results

Mechanical properties of grafted femurs 6 weeks after implantation and the contralateral intact femurs were measured in torsion and reported in Table 6. As expected, PTH treatment improved grafted femur torsional rigidity and strength and failed at with less twist in a more brittle-like fashion indicating the presence of boney union, as opposed to soft callus formation. PTH treatment doubled the torsional rigidity of grafted femurs, returning them to equivalent of intact normal femurs. Yield Torque was also significantly 72% greater in PTH treated grafted femurs, but Ultimate Torque was only 23% greater (not significant). Grafted femurs from saline treated specimens did not fail until reaching a much greater the degree of twist at T_{Ult} than PTH treated specimens. The rate of twist at T_{Ult} for PTH treated specimens was only ⅓ the rotation of control grafted specimens and were similar to the intact contralateral femurs. Work to failure (area under the curve) was not reduced in the saline control group because of the association of low torsional rigidity with failure at greater deformation angles, and similar ultimate torque values. Intact contralateral femurs from the same mice did not show a significant increase in torsional mechanics with 6 weeks of PTH treatment, which is consistent with results from a previous experiment in which intact femurs from rats receiving intermittent did not achieve a significant increase in strength until high dose PTH (100 μg/kg) was given for 8 weeks (Hashimoto, Shigetomi et al. 2007)

TABLE 6
Torsional properties of grafted and contralateral femurs
in mice treated with PTH or saline as control.

| | Sal Graft | | Sal Contra | | PTH Graft | | PTH Contra | |
|---|---|---|---|---|---|---|---|---|
| Tᵤₗₜ (N*mm) | 10.7 | (4.1) | 19.5 | (4.8) | 13.2 | (5.2) | 22.6 | (7.3) |
| T_{yield} (N*mm) | 6.8 | (5.5) | 13.9 | (5.0) ^{†} | 10.5 | (4.2) * | 15.1 | (4.5) ^{†} |
| TR (N*mm²/Rad) | 585 | (408) | 1129 | (362) ^{†} | 1175 | (311) * | 1284 | (205) |
| Twist at Tᵤₗₜ | 0.065 | (0.054) | 0.025 | (0.006) ^{†} | 0.020 | (0.018) * | 0.029 | (0.020) |
| (Rad/mm) | | | | | | | | |
| Work to T_{Yield} | 0.0508 | (0.0615) | 0.134 | (0.090) ^{†} | 0.0687 | (0.0348) | 0.151 | (0.125) ^{†} |
| (Nmm*Rad/mm) | | | | | | | | |
| Work to T_{Ult} | 0.379 | (0.311) | 0.286 | (0.115) | 0.167 | (0.102) | 0.401 | (0.244) |
| (Nmm*Rad/mm) | | | | | | | | |

^{†}p < 0.05 for graft vs. contralateral.
* p < 0.05 for PTH vs saline.

After torsion testing, an x-ray of the specimens was taken to determine the mode of failure as described in (Reynolds, Hock et al. 2007). Despite the trend that PTH-treated specimens had fewer grafted femurs failing due to simple non-union between the graft and host, this was not found to be statistically significant using Fisher's Exact test. Six of the PTH-treated specimens failed in a manner that was not simply graft-pullout from the host, while only 3 of the saline treated specimens appeared to have had some union.

TABLE 7
Grafted femur mode of failure after torsion testing.

| | Pre-Union | Early Union | Mature Union |
|---|---|---|---|
| Saline | 10 | 1 | 2 |
| PTH | 8 | 3 | 3 |

### Correlations Between Micro-CT Parameters and Torsional Properties:

In order to establish associations between micro-CT derived measures and biomechanical outcomes, univariate and multivariate linear regression analysis was performed. The best univariate correlations for each of the mechanical outcomes were as follows: TR vs. UR, r²=0.77; T_{Yield} vs. Bridging, r²=0.62; T_{Ult} vs. PMI_{Min}, r²=0.57. Table 8 shows the Pearson correlation coefficients for all the micro-CT derived measures to the mechanical outcomes.

TABLE 8
Coefficients of determination (R²) for the univariate
linear regression of structural independent variables
vs. mechanical properties TR, T_{Yield} and T_{Ult}.

| | TR | T_{Yield} | T_{Ult} |
|---|---|---|---|
| PMI_{Ave} (mm⁴) | 0.092 | 0.023 | 0.097 |
| PMI_{Max} (mm⁴) | 0.023 | 0.144 | 0.020 |
| PMI_{Min} (mm⁴) | 0.378 * | 0.424 * | 0.567 * |
| BV_{Graft} (mm³/mm) | 0.013 | 0.042 | 0.045 |
| BV_{Callus} (mm³/mm) | 0.288 * | 0.139 | 0.203 * |
| BV_{Intramed} (mm³/mm) | 0.314 * | 0.192 * | 0.161 * |
| BMD_{Callus} (mgHA/cc) | 0.426 * | 0.178 * | 0.072 |
| BMC_{Callus} (mgHA) | 0.202 * | 0.089 | 0.163 * |
| Union Ratio | 0.771 * | 0.589 * | 0.301 * |
| Bridging | 0.403 * | 0.620 * | 0.534 * |

* indicates significance for the two-sided test of the null hypothesis that the slope of the regression line is zero (p < 0.05). The strongest structural predictor for the mechanical outcome is in bold.

### Linear Regressions Between Mechanical Properties and Union Ratio

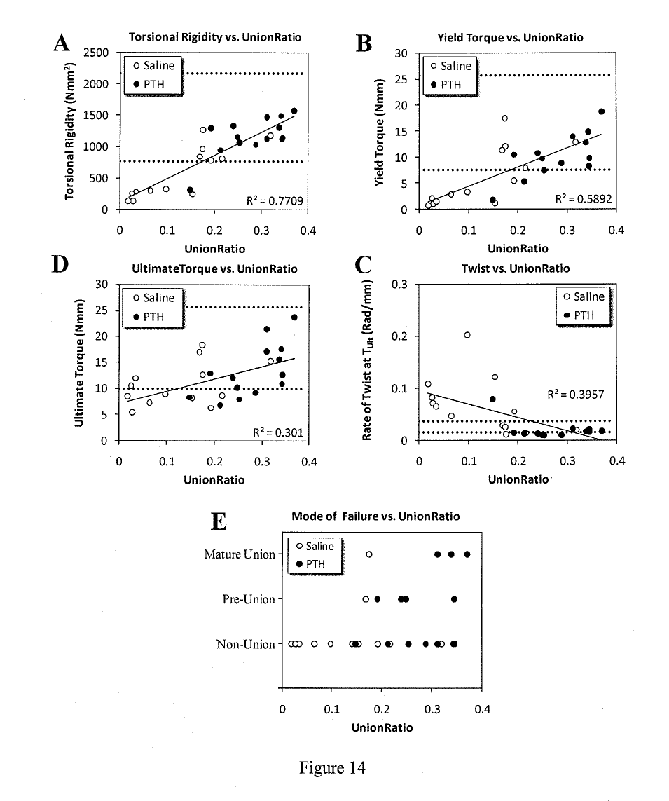

The UR was found to correlate highly with TR (r²=0.77), T_{Yield} (r²=0.59), T_{Ult} (r²=0.30) and inversely with Twist (r²=0.40). Horizontal dotted lines in FIG. 14A-D represent the range of data obtained from the normal contralateral femurs in placebo-treated animals.

### Callus Vascularization Results

Blood vessel analysis was performed using micro-CT imaging after vascular perfusion with a contrast enhancing polymer. Quantification of the vessels within the callus region, as shown in FIG. 17 shows that there was 74% and 88% more vessel volume in saline treated specimens at 4 and 6 weeks respectively (not significant), which was mainly due to an increase in blood vessel diameter (55% greater at 4 weeks p=0.05, 78% greater at 6 weeks, p 0.001).

### Vascularization of Callus in Saline and PTH Treated Animals

Representative vascular analysis via micro-CT imaging of contrast-enhancing vascular profusion agent within the callus after decalcification. Total vascular volume, vessel diameter and vessel number are plotted as mean±SD; n=4 per group. *Significance between treatment (p<0.05).

In both PTH and saline treated animals we observed an interesting phenomenon that a major blood vessel formed down the in the intramedullary canal of the dead graft visible on micro-CT images of vascular-perfused specimens. This is remarkable because despite there being vasculature within the graft, there is little or no sign of revitalization of any other tissue associated by any other cell types. There is neither bone nor hematopoietic marrow in the graft marrow cavity at 4 or 6 weeks in control yet there was a single branch of the femoral artery perforating the host bone shell and passing through the marrow cavity. This is interesting because it would mean there is potential for revitalization of the graft from the interior as well as the periphery.

### Maximum Intensity Projections of Vascular Perfusion Imaging

Maximum intensity projections of micro-CT scans of intercalary allografts in mouse femurs with vascular profusion contrast agent were also performed. Apparent in each image were intramedullary blood vessels inside the graft which by 6 weeks span the entire space from host to host.

### Multivariable Linear Regression Results

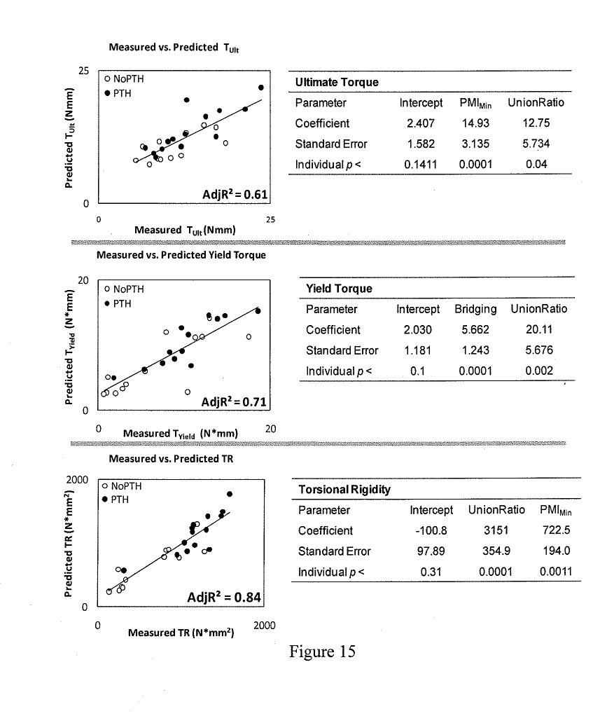

FIG. 15 illustrates a stepwise regression analysis used for variable selection of micro-CT-derived geometric properties such as segmented bone volumes, max, min or average PMIs, Union Ratio, BMD, and host-to-host bridging, resulting in the correlations between micro-CT parameters and torsional ultimate strength, yield strength and rigidity. This analysis yielded correlation equations that could be used to predict functional mechanical outcomes so the coefficients of the measures are given in the tables.

### Discussion

In this study we investigated the use of intermittent teriparatide for standard cortical allografts to determine if the reported anabolic effect in fracture healing also improves allograft bone incorporation. This work shows the anabolic effect of intermittently administered PTH (1-34) effectively stimulates callus formation around bone allografts. This robust callus overcomes the delay in radiographic non-union by 6 weeks, which is an improvement over normal allografts and plays a significant role in improving biomechanical strength and stiffness.

Cartilage formation was increased with PTH treatment, which persisted through 4 weeks after surgery, in which PTH enhanced cartilage formation at the site of bone injury (FIG. 15). This extensive cartilage would then undergo ossification via chondrocyte hypertrophy, thus suggesting that one mode of PTH's effect, which resulted in greater bone volume at 6 weeks, was due to enhanced cartilaginous callus formation.

There was also an improved graft-to-host union ratio apparent on micro-CT in animals treated with PTH which meant that PTH treatment was able to overcome the formation of the fibrous layer that forms around implanted bone grafts, and hinders their incorporation with the host callus.

Side-by-side comparison of histological and radiographic imaging of the union of the callus to allografts indicates that union, in this case, is attributable to callus bone forming directly adjacent to the graft, but not necessarily integrating with that graft tissue via remodeling of the graft initiated from the callus (FIG. 15). To some degree this distinguishes radiographic union from histological union. Here, we found that radiographic union was sufficient to improve bone biomechanics.

We found a preferential enhancement of total bone mineral content of the callus at 6 weeks compared to the enhancement of the systemic skeletal bone mineral content. The ratio of callus BMC_{PTH}:BMC_{Saline} was 1.67 whereas the contralateral intact femur's diaphyseal BMC_{PTH}:BMC_{Saline}=1.17. In addition to the increased cartilaginous callus volume early, the increased surface area during callus formation may be another reason why there is greater improvement in callus bone volume than in the intact contralateral diaphysis. PTH treatment appears to affect multiple stages of bone healing, with enhancement of cartilage early which turns into callus, as well as a greater bone formation rate of the trabecular-like woven bone of the callus.

FIG. 15 shows an obvious proximal non union in the saline control, which corresponds to a Union Ratio of 0.02, which is interpreted as only 2% of that half of the graft is in contact with callus, whereas the PTH-treated specimen in FIG. 15H with UR=0.31 has at least 31% of either half of the graft upon which callus had formed. Across all samples (see, e.g., Table 4), the Union Ratio was greater by 2.8 fold in PTH treated animals. Attaining a level of union that corresponds to a recuperation of the mechanical properties of intact femurs can be identified in the plots of FIG. 14. The intersection of the trend line with the dashed line representing the range of values for normal femurs indicates a threshold at which union could be considered sufficient. With the various mechanical outcome measures, this UR ranged from 0.12 to 0.23 with a mean intersection of 0.18. It can be inferred that achieving that level of union in this mouse model would mean returning the risk of limb fracture to “normal”. Interestingly, this did not correspond to a dramatic shift in the location of failure of these femurs (Table 7). There was a trend that fewer femurs failed in a non-union mode, but this trend was not significant. This suggests that PTH induced robust callus formation adjacent to the graft, but it may not have integrated with the graft.

The result of the improvements in the callus by PTH treatment resulted in stronger graft biomechanics. In fact, it was found that these structural parameters correlate highly with the biomechanical strength and stiffness determined by torsion testing. Univariate regression analysis revealed that as expected, many of the structural bone geometry and density measures correlated strongly with mechanical outcome measures. According to their coefficient of determination (R²) the best of these were the Union Ratio, the host-to-host bridging and PMI_{Min}, (Table 7). Multivariable regression analysis of all the imaging-derived parameters showed that by pairing combinations of those best three predictors the correlation significantly improves, adding 0.14-0.17 to the adjusted coefficients of determination (Adj. R²) (FIG. 20).

The revitalization of the intramedullary canal of the graft with bone is a novel observation and points to another means of revitalizing graft tissue from the inside out. Intramedullary bone formation has not been observed in any of our studies aimed at enhancing mouse allograft bone healing when adjuvant treatments were locally delivered on the periosteal surface of the graft. These studies included the use of rAAV-caAlk2 (Koefoed, Ito et al. 2005), combination rAAV-Vegf and rAAV-RankL (Ito, Koefoed et al. 2005), rAAV-BMP2 and co-engraftment of C9 stem cells (Xie, Reynolds et al. 2007). This increase in BV_{Intramed} was mostly due to increased bone volume fraction and a small increase in the depth of penetration of boney callus into the grafts from both ends. Intramedullary penetration depth was 28% greater in PTH treated animals (2.2±0.7 mm in grafts from PTH-treated animals, 1.7±0.6 mm in grafts from saline-treated animals; p=0.12) but intramedullary callus that was present was densely layered with bone. As on the exterior surface of allografts, the cell types present in the intramedullary canal of allografts from saline treated mice were predominantly fibroblastic, but with PTH treatment the composition of the intramedullary space at the ends of the graft were predominantly osteoblastic cells.

Another novel observation was that a major intramedullary blood vessel was visible on micro-CT images of animals with vascular contrast agent. This was identified as a penetrating branch of the femoral artery that re-bridged, over time, from host to host inside the graft. At 4 weeks the vessel was visible, and by 6 weeks it had bridged the length of the graft in 3 of 4 saline treated specimens, and 4 of 4 PTH treated specimens. From histology it is apparent that the graft intramedullary space is largely void of healing callus or hematopoetic marrow, and instead only sparse fibroblasts and adipocytes. It may have been assumed that there was no nutrient supply to this intramedullary space thus graft regeneration should focus on the periosteal surface, but knowing now that vascular invasion of the graft is present suggests that adjuvant local therapeutics in the intramedullary space should not be ignored as a means of graft revitalization. Studying the revascularization of bone grafts using the intramedullary canal as an indicator of graft revitalization may or may not be appropriate as we found intramedullary vascularization with little or no graft revitalization in both PTH and Saline treated specimens. Resolving the vasculature within the graft material itself may be a better indicator. This deserves further investigation.

Although patients receiving large structural allografts after removing bone neoplasms would be restricted from PTH treatment due to an assumed increased risk of cancer, there are many other uses of bone grafts such as for trauma, joint revision arthroplasty, dental implants, oral surgery, and spinal fusion in which tumors are not involved for which PTH could be utilized without the risk of exacerbating tumor recurrence.

Although a study of morselized autograft for spinal fusion shows early enhancement of osteoclast-related genes and an increase in osteoclast number, and another study of fracture healing in rats showed a significant increase in OC# per fracture callus area, we found that PTH treatment for 6 weeks did not stimulate osteoclastic graft resorption, and thus there was also no increase in revitalization of the graft tissue through remodeling. Contrarily, other studies of fracture calluses show no increase in osteoclast number per bone callus surface area beyond 1 week post-fracture. These discrepancies may be due to the method of counting osteoclasts, whether it is normalized to the cross-sectional callus area, or the perimeter of the mineralized callus surface. To determine whether osteoclastic resorption of the graft can be stimulated via continuous elevation of PTH (or by some other controlled means) should be investigated. PTH could also be used in conjunction with other therapies as a control mechanism to maintain the highest level of graft integrity. The toolbox of systemically administered therapies would then include intermittent PTH for callus formation, short-term bisphosphonates to inhibit osteoclastic resorption and perhaps continuous PTH to stimulate it.

The timing of PTH initiation after injury may be an important control parameter for engineering a rehabilitation regime. We initiated daily saline or PTH injections one week after initial surgery to allow normal hematoma formation to complete before handling the animals daily.

Based on our results we find that the anabolic effect of PTH can significantly improve callus formation from the host around bone grafts and for the first time show a potential solution to improving bone graft-to-host union which would significantly alleviate problems with graft non-unions. This could reduce the need for additional surgical interventions in patients with non-stable constructs.

Although some embodiments of the invention have been described with reference to the disclosed embodiments, those skilled in the art will readily appreciate that the specific examples and studies detailed above are only illustrative of the invention. It should be understood that various modifications can be made without departing from the spirit of the invention.

What is claimed is:

1. A method, of improving an outcome of a bone graft procedure in a patient, comprising:
providing a bone graft to a patient; and
intermittently providing a parathyroid hormone (PTH) to the patient in an amount effective to enhance in or adjacent to the bone graft, relative to a patient not provided the PTH, at least one of callus bone volume, callus mineral content, callus bridging, graft stiffness, graft incorporation, and graft resistance to an applied torque.

2. The method of claim 1, wherein an effective amount is provided by daily injection of the PTH in an amount of at least about 0.1 mg/kg body weight/day.

3. The method of claim 1, wherein an effective amount is provided by daily injection of the PTH in an amount of at least about 0.4 mg/kg body weight/day.

4. The method of claim 1, wherein the PTH is provided for a period of at least 4 weeks.

5. The method of claim 1, wherein the PTH comprises native PTH.

6. The method of claim 1, wherein the PTH comprises PTH (1-34).

7. The method of claim 1, wherein callus bone volume is increased by at least about 75%.

8. The method of claim 1, wherein bone mineral content is increased by at least about 50%.

9. The method of claim 1, wherein ultimate torque is increased by at least about 60%.

10. The method of claim 1, wherein the bone graft provided comprises an autograft.

11. The method of claim 1, wherein the bone graft provided comprises an allograft.

12. The method of claim 1, wherein the PTH is effective to increase bone stiffness.

13. The method of claim 1, wherein the PTH is effective to increase bone brittleness.

14. The method of claim 1, wherein the PTH is effective to result in at least one of a reduced risk of pre-union and early union failure of a graft.

15. The method of claim 1, wherein the PTH is provided in an amount effective to enhance callus bone volume in or adjacent to the bone allograft, relative to a patient not provided the PTH.

16. The method of claim 1, wherein the PTH is provided in an amount effective to enhance callus mineral content in or adjacent to the bone allograft, relative to a patient not provided the PTH.

17. The method of claim 1, wherein the PTH is provided in an amount effective to enhance callus bridging in or adjacent to the bone allograft, relative to a patient not provided the PTH.

18. The method of claim 1, wherein the PTH is provided in an amount effective to enhance graft stiffness, relative to a patient not provided the PTH.

19. The method of claim 1, wherein the PTH is provided in an amount effective to enhance incorporation of the bone allograft, relative to a patient not provided the PTH.

20. The method of claim 1, wherein the PTH is provided in an amount effective to enhance graft resistance to an applied torque, relative to a patient not provided the PTH.
